(19) United States
(12) Patent Application Publication
(10) Pub. No.: US 2021/0052750 A1
(43) Pub. Date: Feb. 25, 2021

(54) ASEPTIC PROCESS FOR AZIDO-FUNCTIONALIZED LIGAND CONJUGATION TO SIZE-ISOLATED MICROBUBBLES VIA STRAIN-PROMOTED AZIDE-ALKYNE CYCLOADDITION

(71) Applicant: The Regents of the University of Colorado, a body corporate, Denver, CO (US)

(72) Inventors: Mark A. Borden, Boulder, CO (US); Connor Slagle, Aurora, CO (US)

(21) Appl. No.: 17/044,266

(22) PCT Filed: Apr. 3, 2019
(86) PCT No.: PCT/US19/25641

§ 371 Date: Sep. 30, 2020

Related U.S. Application Data
(60) Provisional application No. 62/652,258, filed on Apr. 3, 2018.

(51) Int. Cl. A61K 49/22; A61K 47/69; A61K 47/60; A61K 47/54; A61K 47/62

(52) U.S. Cl. CPC A61K 49/223; A61K 47/6925; A61K 47/62; A61K 47/544; A61K 47/545; A61K 47/60

(57) ABSTRACT

The current inventive technology includes system, methods and compositions for the generation of a cloaked microbubble having buried-ligand architecture (BLA) that may allow the cloaked microbubble to circumvent potentially deleterious immunogenic, or other unwanted chemical responses in a host. The current inventive technology may also includes systems and methods to isolate monodisperse size populations of microbubbles for enhanced therapeutic and diagnostic applications.

This application claims the benefit of and priority to U.S. Provisional Patent Application No. 62/652,258, filed Apr. 3, 2018, which is incorporated herein by reference in its entirety.

### GOVERNMENT INTEREST

This invention was made with government support under grant number CA195051 awarded by the National Institutes of Health. The government has certain rights in the invention.

### TECHNICAL FIELD

The present invention includes system, methods and compositions for the generation of cloaked microbubbles through a process of azido-functionalized ligand conjugation via Cu-free click chemistry strain-promoted [3+2] azide-alkyne cycloaddition (SPAAC).

### BACKGROUND

Interest in the use of targeted microbubbles for ultrasound molecular imaging (USMI) has been growing in recent years as a safe and efficacious means of diagnosing tumor angiogenesis and assessing response to therapy. Of particular interest are cloaked microbubbles, which improve specificity by concealing a coupled ligand from blood components until they reach the target vasculature, where the ligand can be transiently revealed for firm receptor-binding by ultrasound acoustic radiation force pulses. Microbubbles are approved in over seventy countries for use in routine ultrasound diagnosis of a wide variety of medical abnormalities of the heart, liver, gastro-intestinal tract, kidneys and other organ systems. At the forefront of this technology are targeted microbubbles, which are being developed for USMI of specific vascular phenotypes, such as inflammation and angiogenesis. Human clinical trials of USMI using microbubbles targeted to biomarkers of tumor angiogenesis were recently reported for noninvasive diagnosis of ovarian, breast and prostate cancers.

Prior reports on microbubble targeting have focused on conventional conjugation chemistries, such as biotin-avidin, maleimide-thiol and carboxyl-to-amine linkages. Unfortunately, these chemistries have significant drawbacks for USMI. The large molecular size of streptavidin increases immunogenicity of the conjugated agent, while unreacted maleimide moieties can lead to cross-reaction with cysteine residues ubiquitously found on serum proteins. Both effects may lead to loss of target specificity, premature clearance and even hypersensitivity. Recently, bio-orthogonal “click” chemistries such as Staudinger ligation, Cu(I)-catalyzed azide-alkyne cycloaddition, and strain-promoted [3+2] azide-alkyne cycloaddition (SPAAC) have been established to provide more efficient and effective ligand conjugation. Of these, SPAAC is the most applicable for USMI because it achieves increased reactivity and stability compared to Staudinger ligation, while avoiding the use of a toxic Cu(I)-catalyst. Commercial reagents for SPAAC are now available, enabling the development and widespread use of new USMI molecular probes.

Another major concern with current targeted microbubbles used for USMI is the potential opsonization of the targeting ligands by blood components. Of particular concern is the ubiquitous complement protein C3, which is converted by nascent C3-convertase enzyme into surface-binding C3b and soluble anaphylatoxin C3a. The protein fragment C3b has an unstable thioester group that may bind to nucleophilic groups present on the targeting ligand, such as hydroxyls. The bound C3b macromolecule on the microbubble surface then further stimulates immunity and diverts specificity from the original target (e.g., an angiogenic biomarker) to C3 receptors present on cells that comprise the mononuclear phagocyte system.

Another drawback of current microbubble formulations that used clinically for contrast-enhanced ultrasound and USMI is their broad size distribution. This is problematic because the circulation persistence and acoustic response of each microbubble to the ultrasound pulse both depend strongly on its size. A resonant microbubble can produce backscatter that is orders of magnitude stronger than its off-resonance counterparts, making it difficult to quantify the number of microbubbles within an imaging voxel. This lack of quantification severely limits the utility of USMI and hinders its prospect for routine clinical use for lesion detection and staging.

Additionally, the acoustic radiation force is maximized when the microbubble is driven at resonance. Off-resonance microbubbles experience less acoustic radiation force and therefore have reduced avidity to the target endothelium. Ideally, to maximize sensitivity, the microbubbles should have a uniform size distribution matched to resonate at the center frequency of the ultrasound imaging probe. The broad particle size distribution is a natural consequence of common manufacturing techniques employed to synthesize microbubble suspensions, such as shaking, sonication or lyophilization/re-suspension. These procedures may involve stochastic physical processes and subsequent Ostwald ripening that yield a polydisperse size distribution.

Others have attempted to address these concerns to vary levels of success. For example, Nagy et. al., describe the use of polymerize shell lipids microbubbles as a delivery vehicle for therapeutic compounds. However, Nagy does to adequately address the immunogenic consequences described above and thus fail to meet the long-felt need for a safe and effective microbubble ligand delivery system. (See U.S. patent application Ser. No. 13/699,298, incorporated herein by reference in its entirety).

As can be seen, there exists a need for an effective microbubble production and ligand-binding and delivery system that addresses the concerns outlined above.

### SUMMARY OF THE INVENTION(S)

One aim of the current inventive technology includes system, methods and compositions for the generation of a cloaked microbubble having buried-ligand architecture (BLA) that may allow the cloaked microbubble to circumvent potentially deleterious immunogenic, or other unwanted chemical responses in a host. Another aim of the current inventive technology includes systems and methods to isolate monodisperse size populations of microbubbles.

Another aim of the current inventive technology includes a BLA system having a hydrated polymer brush architecture that includes a shorter polyethylene glycol (PEG) tether of ˜2000 Da molecular weight that attaches the targeting ligand to an anchoring lipid in the microbubble shell. The tethered ligand may be surrounded by longer PEG chains of ˜5000 Da that, in order to maximize entropy, stratify into an overbrush that conceals the ligand from blood components. The BLA may form a cloaked microbubble.

Another aim of the current inventive technology includes a BLA system having a hydrated polymer brush architecture whereby the cloaked ligand can be transiently revealed by the application of ultrasound through the mechanisms of acoustic radiation force displacement of the cloaked microbubble against the receptor-bearing surface and accompanying surface oscillation of the shell. The ligand tether may be sufficiently flexible to retain the ligand-receptor bond and sustain firm microbubble adhesion to the target endothelium after the acoustic pulse has passed.

Another aim of the current inventive technology includes a BLA system whereby cloaked microbubbles may be configured circulate longer and exhibit greater tumor-targeting specificity in vivo than their uncloaked counterparts.

Another aim of the current inventive technology includes the use of bio-orthogonal SPAAC click chemistry to generate cloaked microbubbles conjugated with one or more ligands. Another aim of the current inventive technology includes kits and methods of using a cloaked microbubbles functionalized by a SPAAC click chemistry mechanism to include a conjugated ligand.

Another aim of the current inventive technology includes kits and methods of using a cloaked microbubble functionalized by a SPAAC click chemistry mechanism to include a conjugated ligand that may be used in ultrasound-based diagnostic and therapeutic technologies.

Another aim of the current inventive technology includes kits and methods of the using a cloaked microbubble functionalized by a SPAAC click chemistry mechanism to include a conjugated ligand that may be used in ultrasound imaging, drug delivery and ultrasound-induced drug delivery.

Another aim of the current inventive technology includes methods of treating an individual comprising administering a cloaked microbubbles functionalized by a SPAAC click chemistry mechanism to include a conjugated ligand to an individual in need of thereof, the microbubble comprising a conjugated ligand having a therapeutic and/or diagnostic effect.

Another aim of the current inventive technology includes a targeted ligand. In some embodiments, the microbubble is conjugated with a ligand and the conjugation is by way of the tethering the ligand to the lipid shell. In some embodiment, the microbubbles comprise a targeting agent. In some embodiments, the targeting agent is specific to a cell surface molecule. In some embodiments, the therapeutic agent within the shell is delivered to a target location by way of the microbubble.

Another aim of the current inventive technology includes the use of bio-orthogonal SPAAC click chemistry to generate cloaked cRGD and A7R peptide-conjugated microbubble against αVβ3 integrin and VEGFR2 biomarkers for angiogenesis expressed on the lumen of tumor neovessels. Such cloaked microbubbles may be produced at optimal resonant size (4-5 μm diameter) for human contrast-enhanced ultrasound imaging (3-7 MHz), and the synthesis process may be sterile and reproducible.

Additional aims of the invention may include one or more of the following embodiments:

1. A method of conjugating a ligand to the surface of a microbubble comprising the steps of:
generating a microbubble having a polymer tether coupled with the surface of said microbubble;
functionalizing at least one ligand to form azido-functionalized ligand; and
conjugating said azido-functionalized ligand to said polymer tether through a click chemistry reaction to form a bioconjugate polymer.

2. The method of embodiment 1 wherein said step of conjugating comprises the step of conjugating said azido-functionalized ligand to said polymer tether through a process of strain-promoted [3+2] azide-alkyne cycloaddition (SPAAC).

3. The method of embodiment 1 wherein said step of conjugating comprises the step of conjugating said azido-functionalized ligand to said polymer tether through a strain-promoted [3+2] azide-alkyne cycloaddition (SPAAC) conjugation reaction between a polymer tether comprising PEGylated, dibenzocyclooctyne-functionalized phosphatidylethanolamine (DSPE-PEG2000-DBCO), and said azido-functionalized peptide ligand to form 1,2,3-triazole linked bioconjugate, wherein said SPAAC reaction is performed in the absence of a copper (Cu) catalyst.

4. The method of embodiment 3 wherein said step of generating a microbubble comprises generating a microbubble having buried-ligand architecture (BLA).

5. The method of embodiment 4 wherein said step of generating a microbubble having BLA comprises generating a microbubble having hydrated polymer brush architecture.

6. The method of embodiment 5 wherein said step of generating a microbubble having hydrated polymer brush architecture comprises a microbubble having bimodal PEGylated surface architecture.

7. The method of embodiment 6 wherein said step of generating a microbubble having bimodal PEGylated surface architecture comprises generating a microbubble having:
a plurality of shorter polyethylene glycol (PEG) molecule forming said polymer tether that attaches said azido-functionalized ligand to an anchoring lipid on said microbubble; and
a plurality of longer PEG chains that stratify into an overbrush that cloaks said azido-functionalized ligand.

8. The method of embodiment 7 wherein said shorter PEG molecule forming said polymer tether has a molecular weight of ˜2000 Dalton (Da), and said longer PEG chains forming said overbrush has a molecular weight of ˜5000 Da.

9. The method of embodiment 2 wherein said azido-functionalized ligand is cloaked by BLA on said microbubble.

10. The method of embodiment 9 wherein said step of functionalizing comprises functionalizing at least one biomarker ligand to form an azido-functionalized biomarker ligand.

11. The method of embodiment 10 wherein said step of functionalizing at least one biomarker ligand to form an azido-functionalized biomarker ligand comprises functionalizing at least one angiogenesis biomarker ligand to form an azido-functionalized angiogenesis biomarker ligand.

12. The method of embodiment 11 wherein said step of functionalizing at least one biomarker ligand to form an azido-functionalized biomarker ligand comprises functionalizing an integrin αvβ3 antagonist (cRGD) ligand to form an azido-functionalized integrin αvβ3 antagonist (cRGD) ligand.

13. The method of embodiment 11 wherein said step of functionalizing at least one biomarker ligand to form an azido-functionalized biomarker ligand comprises functionalizing an VEGFR2 antagonist (A7R) ligand to form an azido-functionalized VEGFR2 antagonist (A7R) ligand.

14. The method of embodiment 9 wherein said step of functionalizing at least one ligand to form an azido-functionalized ligand comprises functionalizing at least one cancer biomarker ligand to form an azido-functionalized cancer biomarker ligand.

15. The method of embodiment 9 wherein said step of functionalizing comprises functionalizing at least one therapeutic ligand to form an azido-functionalized therapeutic ligand.

16. The method of embodiment 9 wherein said step of functionalizing comprises functionalizing at least one diagnostic ligand to form an azido-functionalized diagnostic ligand.

17. The method of embodiment 2 wherein said steps of generating, functionalizing, and conjugating are performed aseptically.

18. The method of embodiment 17 wherein the microbubble that is generated, functionalized and conjugated is aseptic.

19. The method of embodiment 9 and further comprising the step of administrating a therapeutically effective amount of the cloaked microbubble to a patient in need thereof.

20. The method of embodiment 19 and further comprising the step of applying ultrasound radiation to said cloaked microbubble to transiently reveal said azido-functionalized ligand.

21. The method of embodiment 20 wherein said cloaked microbubble is between 4-5 μm in diameter.

22. The method of embodiment 21 wherein said cloaked microbubble is between 3-7 μm in diameter.

23. MB100 A method for generating a conjugated microbubble comprising:
generating a microbubble having a polymer tether coupled with the surface of said microbubble;
functionalizing at least one ligand to form azido-functionalized ligand; and
conjugating said azido-functionalized ligand to said polymer tether through a process of strain-promoted [3+2] azide-alkyne cycloaddition (SPAAC) to form a bioconjugate polymer.

24. A method of conjugating a ligand to the surface of a microbubble comprising the steps of:
performing a Cu-free strain-promoted [3+2] azide-alkyne cycloaddition (SPAAC) conjugation reaction between PEGylated, dibenzocyclooctyne-functionalized phosphatidylethanolamine (DSPE-PEG2000-DBCO), and an azido-functionalized peptide ligand to form 1,2,3-triazole linked bioconjugate, wherein said SPAAC reaction is performed in the absence of a copper (Cu) catalyst.

25. A conjugated microbubble comprising:
a polymer tether coupled with the surface of a microbubble;
at least one azido-functionalized ligand conjugated with said polymer tether through a click chemistry reaction to form a bioconjugate polymer.

26. The conjugated microbubble of embodiment 25 wherein said at least one azido-functionalized ligand conjugated with said polymer tether through a click chemistry reaction to form a bioconjugate polymer comprises at least one azido-functionalized ligand conjugated with said polymer tether through a process of strain-promoted [3+2] azide-alkyne cycloaddition (SPAAC).

27. The conjugated microbubble of embodiment 25 wherein said azido-functionalized ligand conjugated with said polymer tether through a click chemistry reaction to form a bioconjugate polymer comprises least one azido-functionalized ligand conjugated with said polymer tether through a strain-promoted [3+2] azide-alkyne cycloaddition (SPAAC) conjugation reaction between a polymer tether comprising PEGylated, dibenzocyclooctyne-functionalized phosphatidylethanolamine (DSPE-PEG2000-DBCO), and said azido-functionalized peptide ligand to form 1,2,3-triazole linked bioconjugate, wherein said SPAAC reaction is performed in the absence of a copper (Cu) catalyst.

28. The conjugated microbubble of embodiment 27 wherein said microbubble comprises a microbubble having buried-ligand architecture (BLA).

29. The conjugated microbubble of embodiment 28 wherein said microbubble having BLA comprises a microbubble having hydrated polymer brush architecture.

30. The conjugated microbubble of embodiment 29 wherein said microbubble having hydrated polymer brush architecture comprises a microbubble having bimodal PEGylated surface architecture.

31. The conjugated microbubble of embodiment 30 wherein said microbubble having bimodal PEGylated surface architecture comprises a microbubble having:
a plurality of shorter polyethylene glycol (PEG) molecule forming said polymer tether that attaches said azido-functionalized ligand to an anchoring lipid on said microbubble; and
a plurality of longer PEG chains that stratify into an overbrush that cloaks said azido-functionalized ligand.

32. The conjugated microbubble of embodiment 31 wherein said shorter PEG molecule forming said polymer tether has a molecular weight of ˜2000 Dalton (Da), and said longer PEG chains forming said overbrush has a molecular weight of ˜5000 Da.

33. The conjugated microbubble of embodiment 26 wherein said azido-functionalized ligand is cloaked by BLA on said microbubble.

34. The conjugated microbubble of embodiment 33 wherein said azido-functionalized ligand comprises at least one azido-functionalized biomarker ligand.

35. The conjugated microbubble of embodiment 34 wherein said at least one azido-functionalized biomarker ligand comprises at least one azido-functionalized angiogenesis biomarker ligand.

36. The conjugated microbubble of embodiment 35 wherein said at least one azido-functionalized angiogenesis biomarker ligand comprises an azido-functionalized integrin αvβ3 antagonist (cRGD) ligand.

37. The conjugated microbubble of embodiment 35 wherein said at least one azido-functionalized angiogenesis biomarker ligand comprises an azido-functionalized VEGFR2 antagonist (A7R) ligand.

38. The conjugated microbubble of embodiment 34 wherein said at least one azido-functionalized biomarker ligand comprises at least one azido-functionalized cancer biomarker ligand.

39. The conjugated microbubble of embodiment 33 wherein said at least one azido-functionalized ligand comprises at least one azido-functionalized therapeutic ligand.

40. The conjugated microbubble of embodiment 33 wherein said at least one azido-functionalized ligand comprises at least one azido-functionalized diagnostic ligand.

41. The conjugated microbubble of embodiment 26 wherein said conjugated microbubble is generated aseptically.

42. The conjugated microbubble of embodiment 41 wherein said conjugated microbubble is aseptic.

43. The conjugated microbubble of embodiment 33 and further comprising a therapeutically effective amount of the cloaked microbubble is administered to a patient in need thereof.

44. The conjugated microbubble of embodiment 43 wherein said azido-functionalized ligand is transiently revealed through application applying ultrasound radiation to said cloaked microbubble.

45. The conjugated microbubble of embodiment 44 wherein said cloaked microbubble is between 4-5 μm in diameter.

46. The conjugated microbubble of embodiment 45 wherein said cloaked microbubble is between 3-7 μm in diameter.

47. A conjugated microbubble comprising:
a polymer tether coupled with the surface of a microbubble;
at least one azido-functionalized ligand conjugated with said polymer tether through a process of strain-promoted [3+2] azide-alkyne cycloaddition (SPAAC) to form a bioconjugate polymer.

48. A conjugated microbubble comprising:
a polymer tether coupled with the surface of a microbubble, wherein said polymer tether is PEGylated, dibenzocyclooctyne-functionalized phosphatidylethanolamine (DSPE-PEG2000-DBCO);
at least one azido-functionalized peptide ligand conjugated with said polymer tether through a process of strain-promoted [3+2] azide-alkyne cycloaddition (SPAAC), wherein said SPAAC reaction is performed in the absence of a copper (Cu) catalyst

Further scope of the applicability of the presently disclosed embodiments will become apparent from the detailed description and drawing(s) provided below. However, it should be understood that the detailed description and specific examples, while indicating preferred embodiments of this disclosure, are given by way of illustration only since various changes and modifications within the spirit and scope of these embodiments will become apparent to those skilled in the art from this detailed description.

### BRIEF DESCRIPTION OF THE FIGURES

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIGS. 1A-C. Demonstrates an exemplary scheme of Cu-free click chemistry strain-promoted [3+2] azide-alkyne cycloaddition (SPAAC) conjugation reaction between PEGylated, dibenzocyclooctyne-functionalized phosphatidylethanolamine (DSPE-PEG2000-DBCO) (a) and azido-functionalized peptide ligand (c) to form 1,2,3-triazole (blue) linked bioconjugate (b). Peptide ligands (red) include: Integrain αvβ3 antagonist cyclic Arg-Gly-Asp (cRGD) (R1), and VEGFR2 antagonist Ala-Thr-Trp-Leu-Pro-Pro-Arg (A7R) (R2).

FIG. 1D. Cartoon of a perfluorobutane (PFB) size-isolated microbubble suspended in phosphate-buffered saline (PBS) (left) and the cloaked ligand (right). The buried-ligand surface architecture is composed of targeting ligands tethered to the lipid monolayer by short (˜2000 Da) polyethylene glycol (PEG) chains protected by a long (˜5000 Da) shielding PEG overbrush layer. The non-specific complement protein C3 is shown to scale.

FIG. 2. Number-weighted (left) and volume-weighted (right) microbubble population distributions for size-isolated microbubbles (black) and polydisperse microbubbles (red). Typical particle-size distributions (a,b) with population mean diameter (c,d) and span (e,f) box plots (n≥10). Span [(P90-P10)/P50] was found to be significantly different (* denotes p<0.001) between size-isolated microbubbles and conventional polydisperse microbubbles.

FIG. 3. Flow cytometry results of pre-conjugation (black) and post-conjugation (green) fluorescence-tagged microbubbles. Side-scatter vs. forward-scatter profile (a), FL2-A vs. FL1-A filtered light intensity (b), and normalized FL1-A intensity histograms (c) are shown for 4-5 μm size gated (P1, red) microbubbles before and after SPAAC conjugation. FL1-A was found to be significantly different (*, p<0.001) before and after conjugation.

FIG. 4. Microbubble shell microstructure. (a) Shown are greyscale epifluorescent microscopy images (100×, 5 μm scale bar) of two size-isolated microbubbles at different focal depths: (left) mid-line of the bubble and (right) top of the bubble. The DSPE-PEG2000-fluorescein distribution is concentrated in the regions (bright) between solid DAPC lipid-enriched domains (dark); (b) exemplary microbubble shell microstructure showing buried-ligand surface architecture is composed of targeting ligands tethered to the lipid monolayer

FIG. 5. Standard normal variate (SNV) normalized FTIR spectra for azido-functionalized ligands (dashed) and microbubble shell components (solid); (a) spectra for A7R (black, dashed), cRGD (blue, dashed), DAPC (black), DSPE-PEG2000-DBCO (blue), and DSPE-PEG5000 (red), are shown next to (b) spectra for A7R-conjugated (black), cRGD-conjugated (blue) and unconjugated (control, red) microbubbles. Principle Component Analysis (PCA) score plot (c) of the fingerprint region (650-1700 cm−1) for conjugated (black, blue), control (red) microbubbles (hollow shapes) and pure species. Clusters for azido-functionalized ligands (black, solid), lipids (red, solid), conjugated microbubbles (black, dashed) and unconjugated microbubbles (red, dashed) are indicated. Comparative box-plots (d) of A7R-conjugated (black), cRGD-conjugated (blue) and unconjugated (red) microbubbles for major principle components. Scores for conjugated microbubbles were found to be significantly different (p<0.01) than unconjugated microbubbles for all major principle components, thereby confirming ligand conjugation.

FIG. 6. Contrast pulse sequence (CPS, 7 MHz) ultrasound images from the dose escalation tolerability study in canines. Shown are images before cloaked-RGD microbubble injection, at maximum contrast (0.8 mechanical index) and after microbubble elimination (1.9 mechanical index) (left to right) of the kidney (sagittal) at 10-3 (a), 10-2 (b), and 10-1 (c) mL/kg microbubble doses (1×107 microbubbles/mL). The contrast-enhanced ultrasound procedure timeline is presented with indicators for microbubble injection, CPS imaging and size-isolated microbubble (SIMB) destruction modes.

### MODE(S) FOR CARRYING OUT THE INVENTION(S)

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries. The present disclosure is explained in greater detail below. This disclosure is not intended to be a detailed catalog of all the different ways in which embodiments of this disclosure can be implemented, or all the features that can be added to the instant embodiments. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which variations and additions do not depart from the scope of the instant disclosure. Hence, the following specification is intended to illustrate some particular embodiments of the disclosure, and not to exhaustively specify all permutations, combinations, and variations thereof.

The present invention provides for microbubbles. The term microbubbles refers to vesicles which are generally characterized by the presence of one or more membranes or walls or shells surrounding an internal void that is filled with a gas or precursor thereto. In some embodiments, the microbubbles comprise one or more lipids. The term lipids includes agents exhibiting amphipathic characteristics causing it to spontaneously adopt an organized structure in water wherein the hydrophobic portion of the molecule is sequestered away from the aqueous phase. As described below, a microbubble may also contain target ligands, or other therapeutic agents, and/or other functional molecules.

In some embodiments, the microbubble has a diameter size range that is about 3-5 μm. In some embodiments, the microbubble has a diameter size range that is about 1-5 μm. In some embodiments, the microbubble has a diameter size range that is about 4-5 μm. In some embodiments, the microbubble has a diameter size of about 4.5 μm. In another embodiment, the microbubble has a diameter size of about 4 μm or about 5 μm. In one embodiment, the microbubble has a diameter size of greater than 5 μm. In one embodiment, the microbubble has a diameter size of less than 1 μm.

In one aspect the invention provides gas filled microbubbles. In some embodiments the microbubbles comprise one or more gases inside a lipid shell. In some embodiments, the lipid shell comprises one or more polymerizable lipids. In some embodiments, the invention provides gas filled microbubbles substantially devoid of liquid in the interior. In some embodiments, the microbubbles are at least about 90% devoid of liquid, at least about 95% devoid of liquid, or about 100% devoid of liquid.

The microbubbles included in this description may contain any combination of gases suitable for the diagnostic or therapeutic method desired. For example, various biocompatible gases such as air, nitrogen, carbon dioxide, oxygen, argon, xenon, neon, helium, and/or combinations thereof may be employed. Other suitable gases will be apparent to those skilled in the art, the gas chosen being only limited by the proposed application of the microbubbles. In some embodiments, the microbubbles contain gases with high molecular weight and size. In some embodiments, the microbubbles contain fluorinated gases, fluorocarbon gases, and perfluorocarbon gases. In some embodiments, the perfluorocarbon gases include perfluoropropane, perfluorobutane, perfluorocyclobutane, perfluoromethane, perfluoroethane and perfluoropentane, especially perfluoropropane. In some embodiments, the perfluorocarbon gases have less than six carbon atoms. Gases that may be incorporated into the microbubbles include but are not limited to: SF6, CF4, C2F6, C3F6, C3F8 C4F6, C4F8, C4F10, C5F10, C5F 12, C6F 12, (1-trifluoromethyl), propane (2-trifluoromethyl)-1,1,1,3,3,3 hexafluoro, and butane (2-trifluoromethyl)-1,1,1,3,3,3,4,4,4 nonafluor, air, oxygen, nitrogen, carbon dioxide, noble gases, vaporized therapeutic compounds, and mixtures thereof. The halogenated versions of hydrocarbons, where other halogens are used to replace F (e.g., Cl, Br, I) would also be useful.

In some embodiments, microbubbles containing gases with high molecular weight and size are used for ultrasound imaging purposes. Without intending to be limited to any theory, the gases with high molecular weight and size enhance ultrasound scattering.

In some embodiments, innocuous, low boiling liquids which will vaporize at body temperature or by the action of remotely applied energy pulses, like C6F14, are also usable as a volatile confinable microbubble component in the present invention. In some embodiments, the confined gases may be at atmospheric pressure or under pressures higher or lower than atmospheric; for instance, the confined gases may be at pressures equal to the hydrostatic pressure of the carrier liquid holding the gas filled microspheres.

In some embodiments, the microbubbles of the invention comprise a conjugated target ligand or conjugated ligand—the terms being generally interchangeable. A conjugated ligand may include a molecule, macromolecule, or molecular assembly which binds specifically to a biological target.

In some embodiments, a ligand may be one or more molecules which specifically bind to receptors, moieties or markers found on vascular or cancerous cells. In some embodiments, targeting agents are molecules which specifically bind to receptors, moieties or markers found on cells of angiogenic neovasculature or receptors, moieties or markers associated with tumor vasculature. The receptors, moieties or markers associated with tumor vasculature can be expressed on cells of vessels which penetrate or are located within the tumor, or which are confined to the inner or outer periphery of the tumor.

In one preferred embodiment, a ligand that may be conjugated to a microbubble may include a molecule, macromolecule, or molecular assembly which may be coupled to a microbubble, and preferably a cloaked microbubble, through a Cu-free click chemistry strain-promoted [3+2] azide-alkyne cycloaddition (SPAAC) “click” chemistry mechanisms. In another preferred embodiment, a conjugated ligand may include a peptide which may be coupled to a microbubble, and preferably a cloaked microbubble, through a SPAAC click chemistry mechanisms.

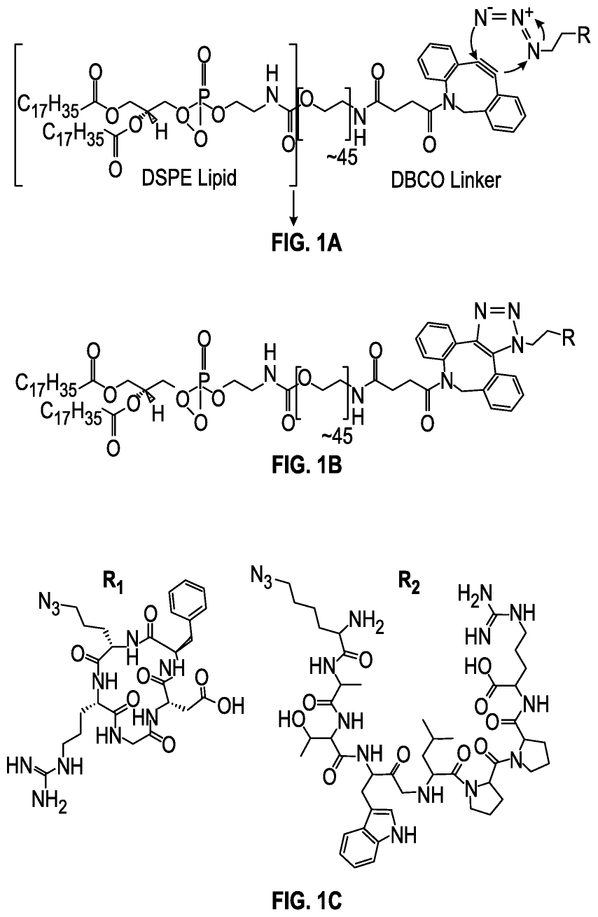

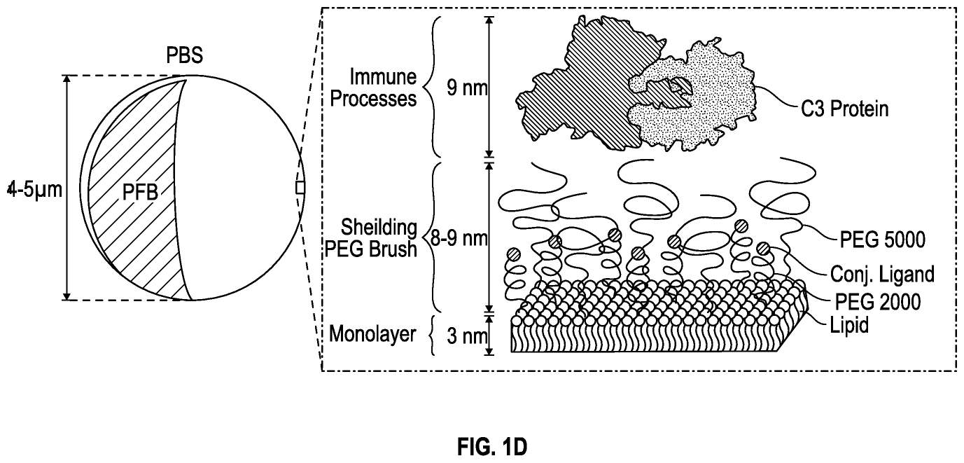

As generally shown in FIG. 1, in some embodiments, a microbubble shell comprises poly(ethylene glycol) (PEG) polymers tethered to a lipid monolayer. Without intending to be limited to any theory, the PEG polymers tethered to the lipids provide colloidal stability against aggregation and steric effects to block binding of opsonizing plasma proteins, which leads to increased lifetime in blood circulation. In some embodiments, the present invention describes microbubbles conjugated with one or more target ligands. In on preferred embodiment shown in FIG. 1D, such conjugated ligands may include PEG-lipid tethered ligands, and may be part of a monodisperse population of microbubbles.

In one embodiment, the invention includes the generation and application of bimodal-brush microbubbles, and preferably a hydrated polymer brush architecture, which may include a bimodal PEGylated surface architecture. In this preferred embodiment, the surface of a microbubble may be modified with a polymer, such as, for example, with PEG. This PEG layer may be bimodal in nature wherein a first population of PEG polymers is of a discrete length, and a second population of PEG polymers is of a different discrete length. One or more ligands may be conjugated with a polymer, such as a PEG polymer that tethered to a microbubble's lipid monolayer. Referring back to FIG. 1, in a preferred embodiment, one or more ligands may be conjugated with a polymer, such as a PEG polymer, that is shorter in length than a second polymer, which may also be of the same, equivalent or different material.

Referring again to FIG. 1 generally, in one preferred embodiment a bimodal-brush microbubble may include a shorter PEG polymer tether, in this instance being of ˜2000 Da molecular weight that may tether the target ligand to an anchoring lipid monolayer. Notably, the ˜2000 Da PEG chains (PEG2000) extend approximately 4 nm above the microbubble's lipid monolayer.

The bimodal-brush microbubble may further include a longer polymer, such as a PEG polymer, that may surround the tethered ligand. In this this embodiment demonstrated in FIG. 1, a tethered ligand may surrounded by longer PEG chains of ˜5000 Da that, in order to maximize entropy, stratify into an overbrush that conceals the ligand from blood components and other unwanted chemical or molecular reactions. In this manner the tethered ligand is “cloaked” by the larger polymers that form the overbrush. Notably, the ˜5000 Da PEG chains (PEG5000) extend approximately 9 nm above the surface of the microbubble's lipid monolayer.

As noted above, a cloaked ligand, preferably a ligand that may bind to one or more receptors in a host, can be transiently revealed by the application of ultrasound through the mechanisms of acoustic radiation force displacement of the cloaked microbubble against the receptor-bearing surface and accompanying surface oscillation of the shell. In one embodiment, the frequency of the ultrasound required to transiently uncover the conjugated ligand from a cloaked microbubble may preferably vary from about 3 to 7 MHz. Additional embodiments may include an optimal frequency of the ultrasound of less than 3 MHz, while still further embodiment require include an optimal frequency of the ultrasound of more than 3 MHz. Additional embodiments may include an optimal frequency of the ultrasound of less than 7 MHz, while still further embodiment require include an optimal frequency of the ultrasound of more than 7 MHz.

The inventive technology includes systems, methods, and compositions to utilize “click” conjugation chemistry to decorate the surface of cloaked microbubbles as part of a sterile and reproducible production process. In one preferred embodiment, the inventive technology includes systems, methods, and compositions to utilize a bio-orthogonal “click” conjugation chemistry to decorate the surface of cloaked 4-5 μm diameter microbubbles as part of a sterile and reproducible production process.

In another exemplary embodiment, ligands, and in particular azido-functionalized ligands may be conjugated to bimodal-brush microbubbles via SPAAC click chemistry. In one preferred embodiment, such conjugated ligand may include one or more therapeutic molecules, such as small peptides or other inhibitors that may be delivered to a discrete tissue or organ to treat and/or diagnose a disease condition. As generally shown in FIG. 1A-D, in one exemplary embodiment, azido-functionalized antagonists for the angiogenic biomarkers αVβ3 integrin (cRGD) and VEGFR2 (A7R) proteins may be conjugated to bimodal-brush microbubbles via SPAAC click conjugation. As demonstrated below, in one embodiment, ligand conjugation to a microbubble may be validated by epifluorescent microscopy, flow cytometry and Fourier-transform infrared spectroscopy. In yet another embodiment, sterility of the cloaked microbubble may also be validated on such novel cloaked microbubbles by bacterial culture and endotoxin analysis.

A therapeutically effective amount of cloaked microbubbles having a select conjugated ligand may be administered to a host, such as an animal, and preferably a mammal or human patient. In this embodiment, a host may receive an initial, repeat or escalating microbubble doses and may experience no pathologic changes in physical examination, complete blood count, and serum biochemistry profile or coagulation panel.

In another embodiment, a therapeutically effective amount of cloaked microbubbles having a select conjugated ligand, and preferably a therapeutic and/or diagnostic ligand may be delivered to a host, and more specifically a host experiencing a disease condition. In this preferred embodiment, a therapeutically effective amount of cloaked microbubbles having a select conjugated ligand that may be delivered to a cancer cell or tumor. The cloaked ligand may be introduced to the cell or tumor by the application of ultrasound through the mechanisms of acoustic radiation force displacement of the microbubble against the receptor-bearing surface and accompanying surface oscillation of the shell.

In another embodiment, a therapeutically effective amount of cloaked microbubbles having containing one or more select conjugated ligand that binds to a biomarker. A biomarker may be associated with a disease condition, for example cancer, as well as a physiological or disease-related process, such as angiogenesis. In one preferred embodiment, a SPAAC click chemistry process may be utilized generate cloaked microbubble having or more anti-angiogenesis peptide ligands. Specifically, a SPAAC click chemistry process may be utilized generate cloaked microbubble having a cRGD and/or A7R peptide-conjugated microbubble against αVβ3 integrin and VEGFR2, which are known biomarkers for angiogenesis expressed on the lumen of tumor neovessels. In this embodiment, the binding of the conjugated anti-angiogenic peptides may both inhibit angiogenesis in a tumor, thus treating a disease condition in a host, as well as allow enhanced visualization and detection of tumor cells in a host through the improved ultrasound visualizations allowed by the presence of the microbubbles at the site of the tumor or cancerous cell. Such enhanced visualization may be accomplished in vivo.

In some embodiments, the invention provides compositions and methods for the diagnosis and/or treatment of a condition. In some embodiments, a cloaked microbubble having one or more conjugated ligands may be used with ultrasound, MRI, or other imaging techniques. Ultrasound visualization of cloaked microbubble having one or more conjugated ligands may also be used to identify and locate solid tumors, angiogenesis activity associated with a disease state such as cancer.

All publications, patents, and patent applications mentioned in this specification are herein incorporated by reference to the same extent as if each individual publication, patent, or patent application was specifically and individually indicated to be incorporated by reference.

As used herein, the term “ligand” means any small molecular weight (<5000 Da) molecule that may be functionalized with an azido group and conjugated to the surface of a microbubble and cloaked for molecular imaging.

“Microbubbles” and “bubbles” are used interchangeably herein to refer to a gas core surrounded by a lipid membrane, which can be either a monolayer or a bilayer and wherein the lipid membrane can contain one or more lipids and one or more stabilizing agents. A microbubble may also mean a liposome and/or a micelle.

A “conjugated microbubble” means a microbubble that is coupled with at least one ligand. A “cloaked microbubble” means a microbubble having buried-ligand architecture (BLA).

A “target ligand,” or “ligand” means a molecule or compound that can be chemically modified by addition of an azide or alkynyl group, such as small molecules, natural products, or biomolecules (e.g., peptides or proteins), such as exemplary ligands cRGD and A7R. A “target ligand,” or “ligand” further means a molecule or compound that that may be conjugated through a SPAAC click chemistry mechanism to a microbubble. Example ligands may include, but not be limited to, drug, a chemical, antibodies, ligands, proteins, peptides, carbohydrates, vitamins, nucleic acids, or combinations thereof.

A “bioconjugate” or “bioconjugate ligand” means a ligand conjugated with a polymer tether.

The term “aseptic” means free from contamination caused by harmful bacteria, viruses, or other microorganisms, or sufficient free from contamination caused by harmful bacteria, viruses, or other microorganisms such that an aseptically produce microbubble or aseptic microbubble may be administered therapeutically to a host, such as a human or animal host.

As used herein, the general term biological marker (“receptors” “biomarker” or “marker” “moieties”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process. A target molecules or markers, and their corresponding interaction with a cloaked microbubble conjugated with a ligand, may be used for diagnostic and prognostic purposes, as well as for therapeutic, drug screening and patient stratification purposes (e.g., to group patients into a number of “subsets” for evaluation), as well as other purposes described herein.

The present invention includes all compositions and methods relying on correlations between the reported markers, cloaked microbubbles, and the therapeutic effect of cancer cells. Such methods include methods for determining whether a cancer patient or tumor is predicted to respond to administration of a therapy, as well as methods for assessing the efficacy of a therapy. Additional methods may include determining whether a cancer patient or tumor is predicted to respond to administration of a therapy. Further included are methods for improving the efficacy of a therapy, such as a cancer therapy, by administering to a subject a therapeutically effective amount of cloaked microbubble having one or more conjugated ligands that binds to, alters the activity of a biomarker, such as an angiogenesis markers such as integrin αVβ3, of VEGFR-2. In this context, the term “effective” is to be understood broadly to include reducing or alleviating the signs or symptoms of a disease condition, improving the clinical course of a disease condition, enhancing killing of cancerous cells, or reducing any other objective or subjective indicia of a disease condition, including indications of responsiveness to a treatment or non-responsiveness to a treatment, such as chemotherapy or radiation treatment. Different therapeutic microbubbles, doses and delivery routes can be evaluated by performing the method using different administration conditions.

The target ligands and cloaked microbubble compositions of the invention are useful for determining if a therapy, such as chemotherapy or radiation, may be an effective treatment for cancer or other disease condition. The target ligands and cloaked microbubble compositions of the invention are useful for predicting the outcome or determining the effectiveness of therapy in multiple cancer types, including without limitation, bladder cancer, lung cancer, head and neck cancer, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, memngioma,pinealoma, ependymoma, craniopharyngioma, epithelial carcinoma, embryonal carcinoma, squamous cell carcinoma, base cell carcinoma, fibrosarcoma, myxoma, myxosarcoma, liposarcoma, chondrosarcoma, osteogenic sarcoma, and leukemia.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below.

The term “therapeutically effective amount” means an amount effective to produce a detectable physiological effect, such as ligand binding to a marker, delivering a ligand to a cell or tissue, or enhancing ultrasound imaging and the like.

Optionally, the therapeutic methods and ligand/microbubble compositions of the present invention may be combined with other anti-cancer therapies and other therapies. Examples of anti-cancer therapies include traditional cancer treatments such as surgery and chemotherapy, as well as other new treatments.

The term “including” is used herein to mean, and is used interchangeably with, the phrase “including but not limited to.” The term “or” is used herein to mean, and is used interchangeably with, the term “and/or,” unless context clearly indicates otherwise.

As used herein, the following abbreviations mean: A7R, Lys(Azide)-Ala-Thr-Trp-Leu-Pro-Pro-Arg; BLA, buried-ligand architecture; CPS, contrast pulse sequencing; cRGD, cyclo[Arg-Gly-Asp-D-Phe-Lys(Azide)]; FTIR, Fourier-transform infrared spectroscopy; PBS, phosphate-buffered saline; PCA, Principle Component Analysis; PEG, polyethylene glycol; PFB, perfluorobutane; PNP, peak-negative-pressure; PSD, particle-size distribution; microbubble, size-isolated microbubble; SNV, standard normal variate; SPAAC, strain-promoted [3+2] azide-alkyne cycloaddition; UCA, ultrasound contrast agent; USMI, ultrasound molecular imaging; VEGFR2, vascular endothelial growth factor receptor 2; DSPE-PEG2000-DBCO, 1,2-distearoyl-sn-glycero-3-phosphoethanolamine-N-[dibenzocyclooctyl (polyethylene glycol)-2000]; DSPE-PEG5000, 1,2-Distearoyl-sn-glycero-3phosphoethanolamine-N-[methoxy (polyethyleneglycol)-5000]; DAPC, 1,2-Diarachidoyl-sn-glycero-3-phosphocholine.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Development of Bio-orthogonal Conjugation Chemistry to Generate Novel Cloaked Microbubble

The present inventors demonstrate a novel bio-orthogonal conjugation chemistry that allows sterile, repeatable production of cloaked microbubbles, which in one preferred embodiment may be used for USMI of tumor neovessels. Certain components of an exemplary cloaked microbubble are demonstrated. In this preferred embodiment, a perfluorobutane (PFB) gas core of 4-5 μm diameter may be coated with a approximately 3-nm thick lipid monolayer and suspended in an aqueous medium.

### Example 2: Development of Bimodal PEGylated Surface Architecture

As further shown in FIG. 1D, the present inventors demonstrate a cloaked microbubble having a bimodal PEGylated surface architecture configured in this embodiment to minimize interactions between a peptide ligand and blood components during systemic circulation. The engineered bimodal PEGylated surface architecture is shown with an exemplary complement protein C3, a major opsonin of the innate immune system that can alter ligand specificity and tag the microbubble for premature clearance by the mononuclear phagocyte system. Self consistent field theory of bimodal brushes predicts that the 2000 Da PEG chains (PEG2000) extend 4 nm above the surface, while the 5000 Da PEG chains (PEG5000) extend 9 nm. As shown in FIG. 1D, this embodiment provides an over-brush layer that is sufficiently thick to hinder C3b interaction with the conjugated peptide ligand. At the same time, the architecture may be designed to allow firm ligand receptor binding during acoustic radiation force pulsing with ultrasound in the target tissue. Additional biocompatible water-soluble polymers in addition to PEG that may be used in the invention include, but are not limited to: N-(2-hydroxypropyl)methacrylamide (HPMA) copolymers, and polyglutamate.

### Example 3: SPAAC Ligand Conjugation Reaction Scheme

An exemplary scheme for SPAAC ligand conjugation reaction is presented in FIG. 1. In this embodiment, surface embedded dibenzocyclooctyne-functionalized, PEGylated, phosphatidylethanolamine (DSPE-PEG2000-DBCO) was reacted with an azido-functionalized ligand (FIG. 1a) to form a resultant bioconjugate lipid (FIG. 1b). Either cyclic Arg-Gly-Asp (cRGD) (R1) peptide, a known αVβ3 integrin antagonist, or Ala-Thr-Trp-Leu-Pro-Pro-Arg (A7R) (R2) peptide, a known VEGFR2 antagonist, were conjugated to microbubbles via this reaction mechanism. These ligands target the microbubbles to neovessels associated with tumor angiogenesis, thereby allowing USMI scans to diagnose tumors and assess their response to therapy.

### Example 4: Population Distributions for Size-isolated Microbubbles and Polydisperse Microbubbles

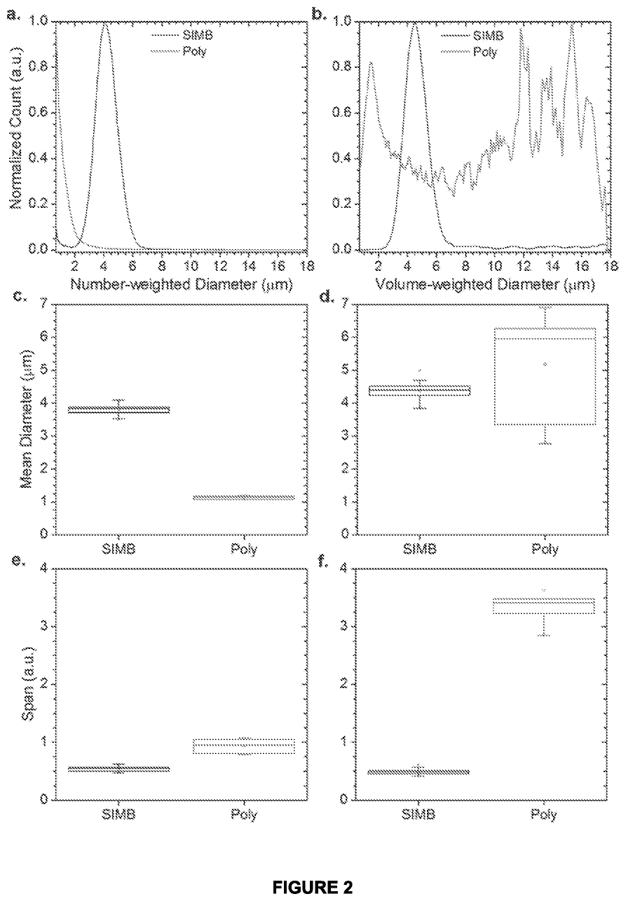

Generally referring to FIG. 2, size-isolated 4-5-μm diameter microbubbles are compared to conventional polydisperse microbubbles. Normalized number-weighted and volume-weighted particle size distributions (FIG. 2a,b) are shown with resulting mean diameter (FIG. 2c,d) and span (FIG. 2e,f) box plots. Notably, span is a variation measure of a particle size distribution, defined as the difference between the 90th percentile and 10th percentile, divided by the 50th percentile (median). The size distribution, mean diameter and span for the size-isolated microbubbles were each found to be significantly different (p<0.001) with a non-parametric Mann-Whitney U-test compared to polydisperse microbubbles produced by the conventional agitation method. The effect of microbubble processing on the volume-weighted distribution can be seen with the polydisperse sample in FIG. 2b. Prior studies on microbubble imaging and targeted drug delivery have shown that the volume-weighted distribution is a more useful dosage unit than the number weighted distribution, as it provides a linear dose-response onto which different microbubble sizes collapse. Overall, the mean diameter and span for both number-weighted and volume-weighted distributions were shown to be repeatable due to the low spread of data compared to polydisperse microbubbles. Significantly, a narrow size distribution and size reproducibility is important for USMI scans, as the radiation force effects and the acoustic backscatter intensity are strongly dependent on microbubble size.

### Example 5: Flow Cytometry Results of Pre-conjugation and Post-conjugation Fluorescence-tagged Microbubbles

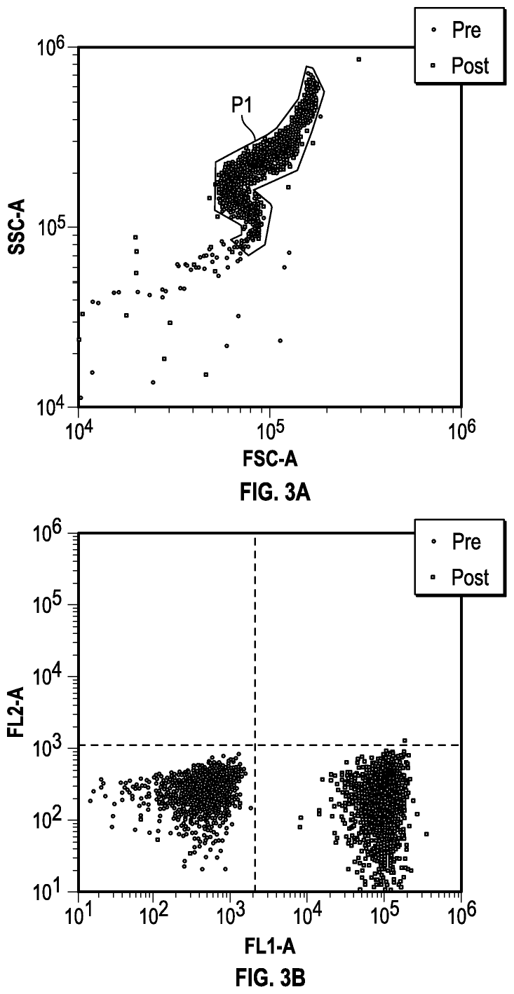

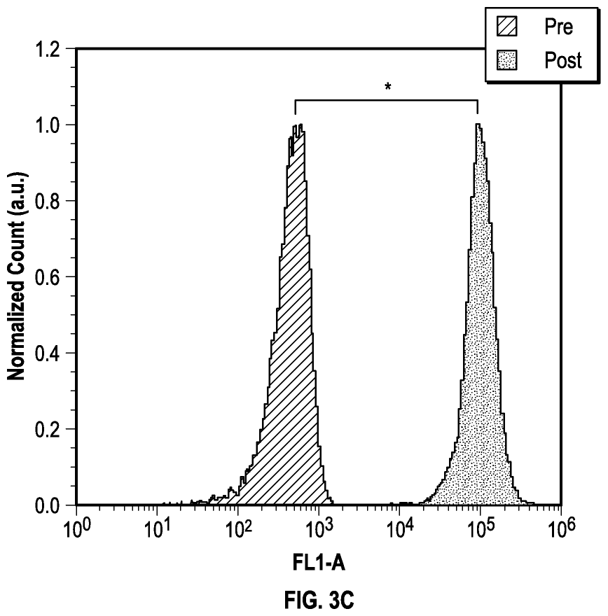

A fluorophore (Atto 488) was conjugated to the microbubble by SPAAC to examine population characteristics and individual particle microstructure. Population characteristics are shown with flow cytometry in FIG. 3. As demonstrated in FIG. 3a, size-gated microbubbles were analyzed before and after conjugation with Atto 488. As demonstrated, the side-scatter versus forward-scatter profiles shows a serpentine-curve characteristic of microbubbles with no change in microbubble size due to the conjugation reaction. As further shown in FIG. 3b, red-filtered (FL2-A) emission light intensity remained constant, while green-filtered (FL1-A) light increased post conjugation.

Generally referring to FIG. 3c, FL1-A intensity versus normalized count showed a clear increase in fluorescence intensity post-conjugation (p<0.001). The increase in fluorescence post-conjugation with no change in physical size or shape, i.e. scatter profile, indicates successful SPAAC conjugation. The serpentine pattern is a consequence of the nonlinear optical scatter of the microbubbles in the flow cytometer beam waist, and indicates both microbubble size and granularity (i.e., presence of surface irregularities such as lipid folds). Both parameters may be useful to control for repeatable and effective USMI, as size affects the radiation force effects and acoustic backscatter, while surface anomalies leading to increased microbubble deformation may affect adhesion efficiency and immunogenicity.

### Example 6: Microbubble Shell Microstructure

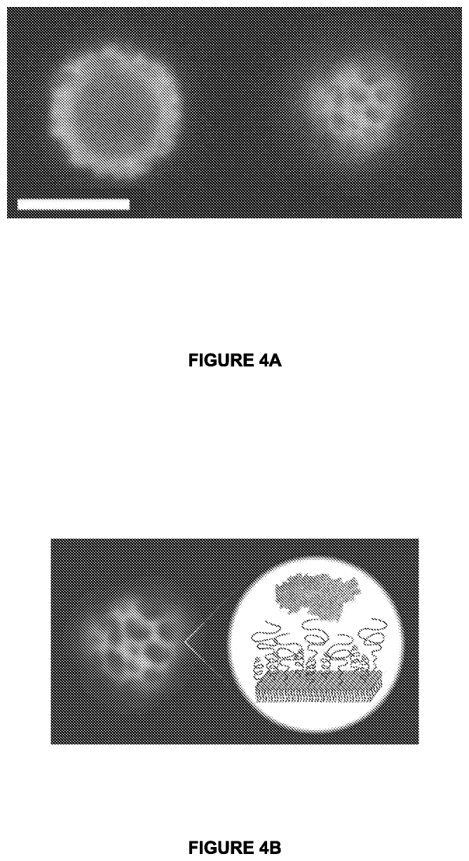

The surface microstructure of individual microbubbles was analyzed by epifluorescent microscopy. FIG. 4 shows greyscale images of two microbubbles at different focal planes. At the mid-bubble focal plane (left), the bright fluorescence intensity of the microbubble surface at the periphery can be contrasted to that of the darker core. The lipid shell exhibited lateral microstructure, with dark domains surrounded by fluorophore-rich interdomain region. These domains can be seen more clearly when the microscope focus is located near the top of the microbubble (right). Similar microstructures were observed for all of the fluorescently tagged microbubbles. The distribution of fluorescence is consistent with prior reports of lateral phase separation between the DAPC matrix lipid and the DSPE-PEG groups. As ligand distribution may ultimately affect ligand-receptor binding efficiency and immunogenicity, such a reproducible novel microstructure is expected to be advantageous.

### Example 7: Standard Normal Variate (SNV) Normalized FTIR Spectra for Azido-functionalized Ligands and Microbubble Shell Components

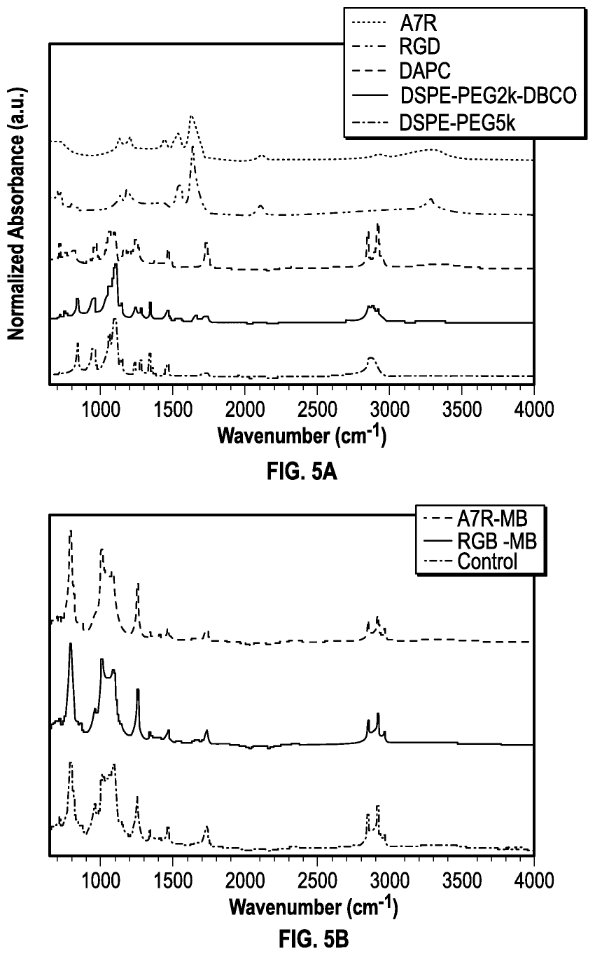

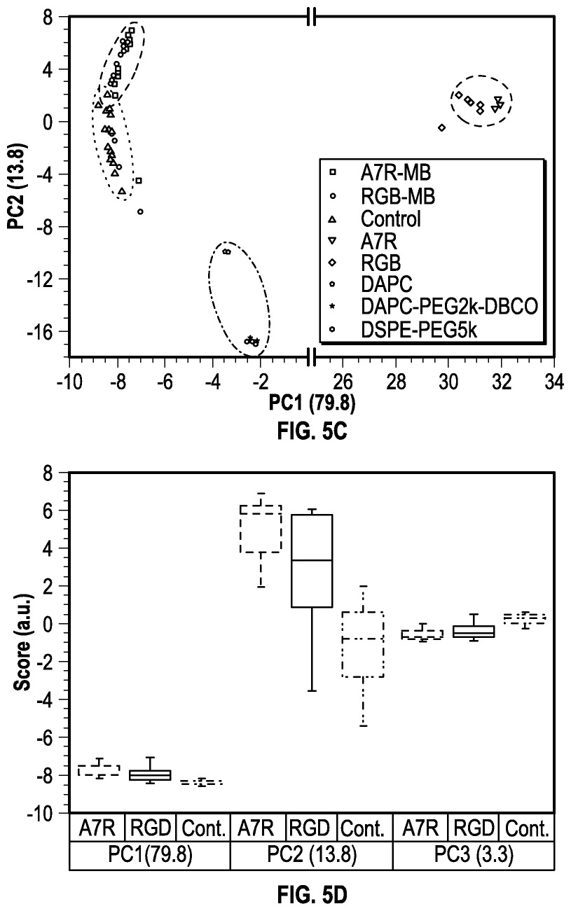

SPAAC conjugation of the targeting peptides was validated by Fourier-transform infrared spectroscopy (FTIR), shown in FIG. 5. Standard normal variate (SNV) normalized FTIR spectra for microbubble component species and azido-functionalized ligands are shown in FIG. 5a, with spectra of ligand-conjugated and control microbubbles in FIG. 5b. SNV normalization reduces sample variability by centering the spectra; normalizing to the mean and standard deviation. After normalization, species-specific absorption bands can be identified and compared to other species. For instance, lipid-based molecules such as DAPC, DSPE-PEG2000-DBCO and DSPE-PEG5000 share aliphatic absorption bands (CH2-bending, ˜1470 cm⁻¹; symmetric CH2-stretching, ˜2850 cm⁻¹; anti-symmetric CH2-stretching, 2920 cm⁻¹), while PEGylated molecules share a sharp C—O stretch absorption band at ˜1090 cm⁻¹. Similarly, heavy amino acid absorbance is observed from ˜1100 cm⁻¹ to ˜1700 cm⁻¹ in both ligands.

Although these peaks are apparent in the species spectra, they become convoluted when measuring microbubble samples. Principle Component Analysis (PCA) was performed on the fingerprint region (650-1700 cm⁻¹) the microbubble spectra as well as the pure species spectra to reduce the dimensionality of the data to orthogonal principle components (PCs). Spectra for pure species and microbubble samples were scored against the first three PCs (PC1, PC2 and PC3) and plotted to identify groups of similar spectra. As shown in FIG. 5c, when PC2 was plotted against PC1, groups of spectra with similar characteristics could be identified. Compounds heavily weighted by lipids (red, solid) scored significantly different than compounds heavily weighted by amino acids (black, solid). Microbubble samples have properties represented by both extremes. Both unconjugated (red, dashed) and conjugated (black, dashed) microbubbles had similar PC1 character, while conjugated microbubbles scored higher in PC2. Quantitatively, conjugated microbubble scores were significantly different (Mann-Whitney, p-value<0.01) than unconjugated microbubble scores in PC1, PC2 and PC3 as demonstrated in FIG. 5d. This PCA analysis therefore confirmed to the present inventors that the peptide ligands were successfully conjugated to the microbubble surface, even with the bimodal PEG brush architecture.

Additional endotoxin analysis and bacterial cultures were performed by the present inventors to validate aseptic production. Produced microbubbles contained only ˜10% of the recommended endotoxin limit, and showed negligible growth on bacterial culture plates.

### Example 8: Contrast Pulse Sequence (CPS, 7 MHz) Ultrasound Images From the Dose Escalation Tolerability Study in Canines

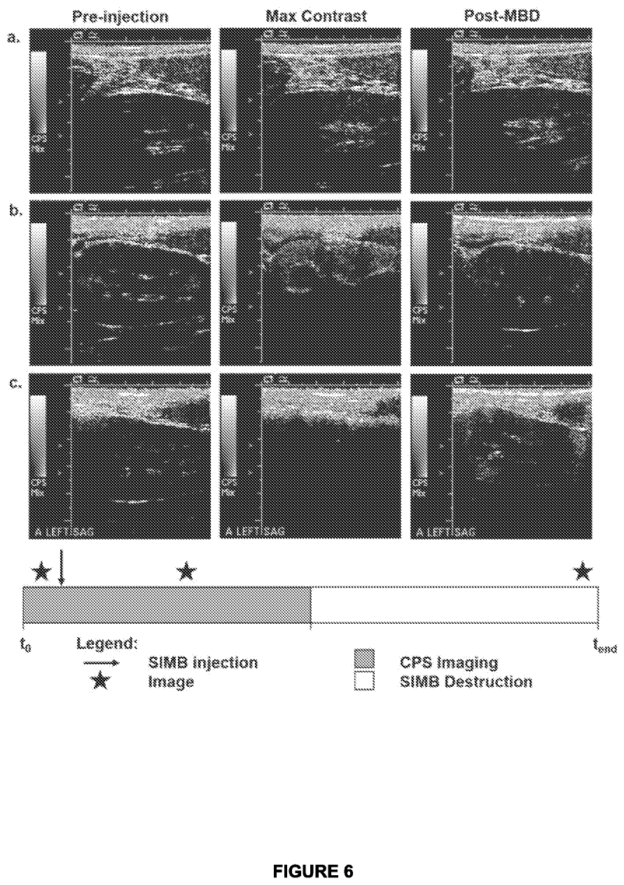

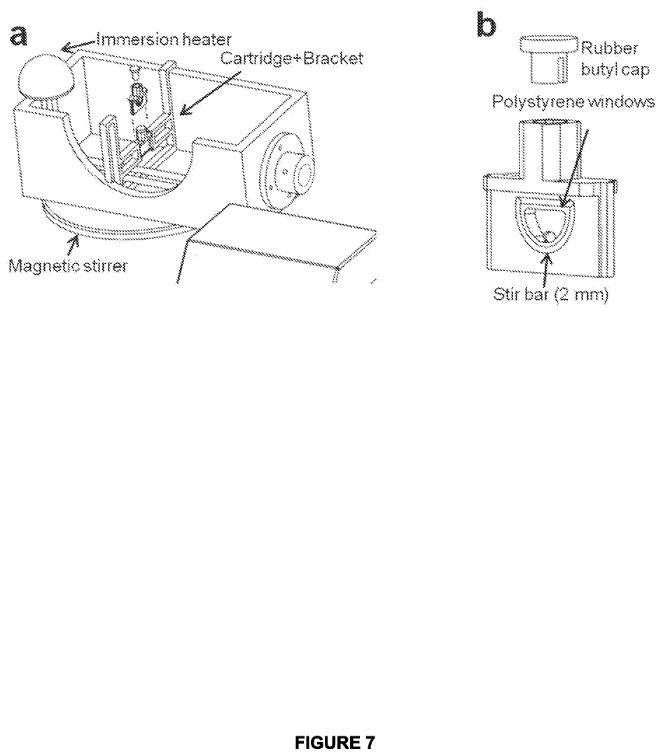

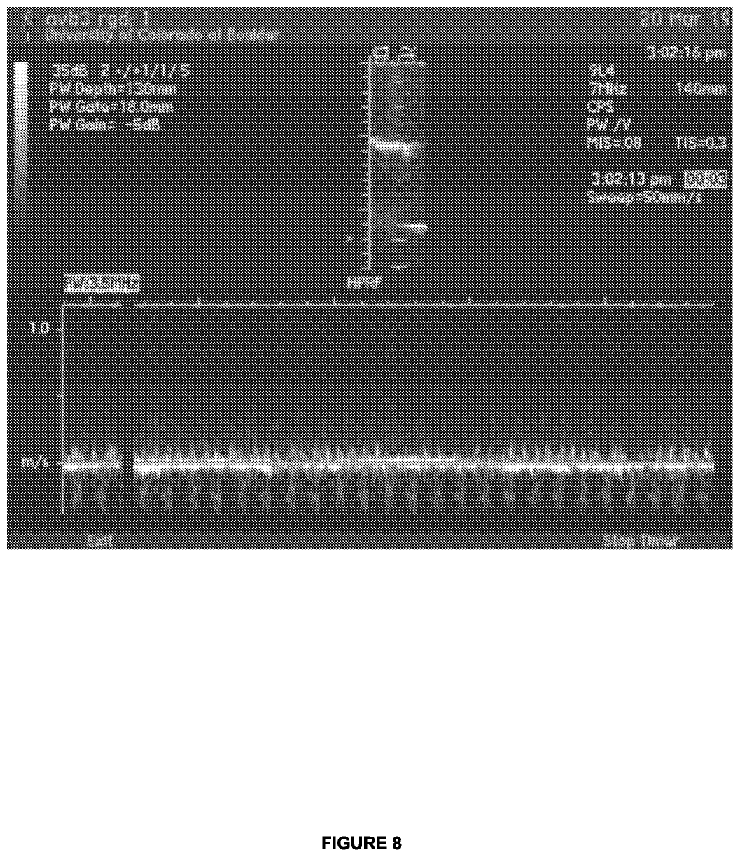

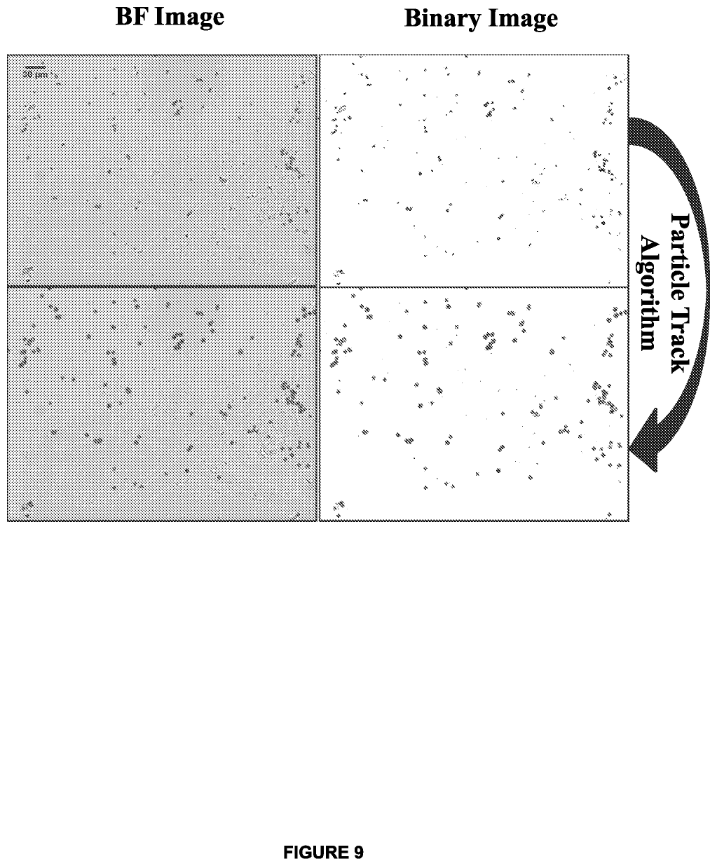

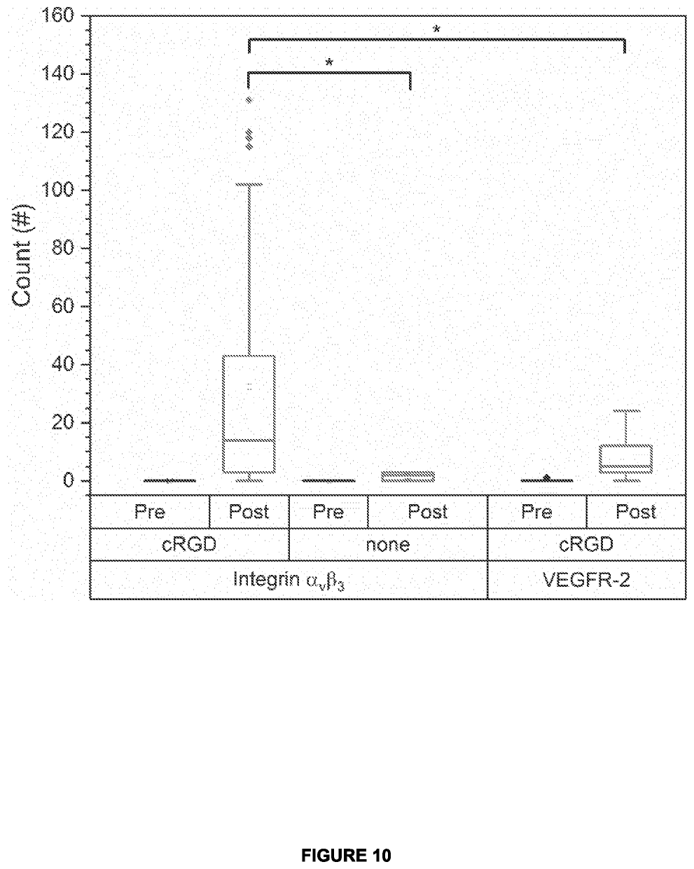

A dose-escalation tolerability study was performed by the present inventors to test the safety of cloaked microbubbles in laboratory beagles. Three laboratory beagles had weekly injections of microbubbles with subsequent ultrasound imaging for three weeks. The dosage increased in 1-log increments from 1 log below our target dose of 0.01 mL/kg (1.0×107 microbubbles/kg) to 1 log above. FIG. 6 shows three contrast-pulse sequence (CPS, 7 MHz) ultrasound images of the kidney for one of the canine subjects (left, sagittal) at each of the three concentrations (top) along with a study timeline (bottom). Images from similar time points were captured for comparison between the low (FIG. 6a), target (FIG. 6b) and high (FIG. 6c) concentration doses and demarcated on the timeline. Images (left to right) were captured prior to microbubble injection, at maximum contrast, and after a period of microbubble elimination from the blood pool. Pre-injection and maximum contrast images were captured under low-intensity (0.80 mechanical index) ultrasound while post-microbubble images were captured after a prolonged insonation at high-intensity (1.9 mechanical index).

An increase in the characteristic non-linear backscatter intensity can be seen at all administration dosages and all low-intensity ultrasound time points. During the high-administration dose, the microbubbles in the kidney significantly attenuated the ultrasound signal, reducing the penetration of ultrasound and resulting in poor-quality images. This effect should be avoided in USMI scans, as shadowing may reduce both radiation force effects and acoustic backscatter intensity of adherent microbubbles. After 2.5 min of imaging at high-intensity, the non-linear acoustic intensity returned to baseline, indicating complete microbubble elimination from circulation. No clinically significant changes in vital signs or any measured clinic-pathological parameter were observed at any time following dosing. This indicates that the cloaked microbubbles are safe and non-immunogenic as injected for USMI in canines. Canines provide a valid and robust preclinical platform for translation to humans, as they are of similar size and present similarly spontaneous, heterogeneous tumors, such as soft tissue sarcomas.

### Example 9: Simulated In Vivo Binding of cRGD/A7R Tagged Size-isolated Microbubbles (SIMBs) to Corresponding Recombinant Proteins

The present inventors demonstrated in vitro binding of select conjugated target ligands to corresponding recombinant proteins. Specifically, cRGD, an integrin αvβ3 antagonist, was conjugated to a cloaked microbubble through Cu-free click chemistry strain-promoted [3+2] azide-alkyne cycloaddition (SPAAC) conjugation reaction between a polymer tether, in this example a PEGylated, dibenzocyclooctyne-functionalized phosphatidylethanolamine (DSPE-PEG2000-DBCO), and an azido-functionalized peptide ligand to form 1,2,3-triazole linked bioconjugate as shown in FIG. 1 (R1). In addition, A7R, a VEGFR-2 antagonist was conjugated to a cloaked microbubble through Cu-free click chemistry strain-promoted [3+2] azide-alkyne cycloaddition (SPAAC) conjugation reaction between PEGylated, dibenzocyclooctyne-functionalized phosphatidylethanolamine (DSPE-PEG2000-DBCO), and azido-functionalized peptide ligand to form 1,2,3-triazole linked bioconjugate as shown in FIG. 1 (R2).

A population conjugated microbubbles were introduced to the apparatus shown in FIG. 7, which generally demonstrates a modified temperature controlled water/bath immersions having a submerged 9L4 array transducer. A quantify of 200 μl of cRGD and A7R conjugated microbubbles were introduced to a immersible sample cartridge. The 9L4 transducer is placed approximately 5 cm away from the sample cartridge, which is meant to simulate in vivo conditions. Specifically, the present inventors employed: a US machine: Siemens Sequoia C512, and a 9L4 transducer with an approximate 5 cm penetration depth, and an applied sonication scheme as described below. Next, controlled protein incubation was allowed to occur at 37° C. for 1 hr with 100 ug/ml receptor protein. During this incubation, integrin αvβ3 and VEGFR-2 was adsorbed to the to cartridge walls to simulate blood vessel wall.

As generally shown in FIG. 8, the present inventors next determined the Acoustic Radiation Force (ARF) parameters of the sample through the application of a High-pulse repetition frequency (HPRF) pulse-wave operation. This HPRF utilizes a 50% duty cycle for 3 minutes (10 s on/10 s off). Utilizing this technique, ARF should push SIMBs in contact with back wall of sample cartridge, exposing the conjugated ligands to the adsorbed protein. Moreover, correctly tagged SIMBs should bind and tether to the back wall—once inverted, they will not rise due to buoyance while untethered/incorrectly tagged SIMBs will rise to top of the cartridge.

The sample cartridge was removed and image capture was taken. Notably, prior to image capture the sample cartridge may be rotated 90° after insonication, with far wall oriented on bottom and close wall oriented on the top of cartridge. As shown in FIG. 9, a bright Field (BF) microscope image is captured and the image is images undergoes an binary adjustment to enhance contrast. A particle tracking particle tracking algorithm is applied to identify and track SIMBs and a count is average for each treatment.

Generally referring to FIG. 10, the cRGD tagged SIMBs bound to adsorbed integrin αVβ3 more than untagged SIMBs, demonstrating successful binding under simulated in vivo conditions. In addition, cRGD tagged SIMBs bound to adsorbed integrin αVβ3 more than to VEGFR-2, further demonstrating successful binding under simulated in vivo conditions. Each measurement was statistically significant (p-value<0.005) with a non-parametric Mann-Whitney test.

### Example 10: Materials and Methods

Materials. 1,2-Diarachidoyl-sn-glycero-3-phosphocholine (DAPC) and 1,2-distearoyl-sn-glycero-3-phosphoethanolamine-N-[dibenzocyclooctyl(polyethylene glycol)-2000] (DSPE-PEG2000-DBCO) were purchased from Avanti Polar Lipids (Alabaster, Ala.). 1,2-Distearoyl-sn-glycero-3-phosphoethanolamine-N-[methoxy(polyethylene glycol)-5000] (DSPE-PEG5000) was purchased from NOF America (White Plains, N.Y.). All lipids were purchased with purity >99% by weight. These lipids are known to produce relatively stiff, and long-circulating microbubbles. Cyclo[Arg-Gly-Asp-D-Phe-Lys(Azide)] (>99%) (cRGD) was purchased from Peptides International (Louisville, Ky.). Lys(Azide)-Ala-Thr-Trp-Leu-Pro-Pro-Arg (>95%) (A7R) was purchased from Genscript (Piscataway, N.J.). Azido-functionalized Atto 488, HPLC-grade chloroform (>99.9%) and HPLC-grade methanol were purchased from Sigma-Aldrich (St. Louis, Mo.). Lipids, peptides and fluorinated dyes were stored in lyophilized form at −20 ° C. until use. Decafluorobutane (>99%) (PFB) was purchased from Fluoromed (Round Rock, Tex.). Reagent-grade isopropyl alcohol (70% v/v) and phosphate buffered saline (PBS) were purchased from Fisher Scientific (Pittsburgh, Pa.). ISOTON® II diluent was purchased from Beckman Coulter (Brea, Calif.). Filtered, deionized water (DI) (0.02 μm, 18.2 MΩ-cm) was produced using the Direct-Q® Millipore Sigma water purification system (Burlington, Mass.).

Lipid Suspension Preparation. A 2.0 mg/mL lipid suspension was used to generate microbubbles. The suspension was produced in 100-500 mL batches using a rotary evaporator (Model R-100, Büchi Corp., New Castle, Del.). To make the suspension, a mixture of DAPC, DSPE-PEG5000 and DSPE-PEG2000-DBCO (18:1:1 molar ratio) was dissolved in chloroform (25 mL for every 100 mL of final suspension). The chloroform suspension was loaded by vacuum into the rotary evaporator and operated at 40° C. and 474 mbar for 4 h. The heat source was powered off and the lipid film was dried at 474 mbar for 15-18 h. Sterilized (Steam, 30 min at 250° C. per 500 mL), filtered (0.02 μm) 1× PBS was loaded by vacuum into the rotary evaporator and initiated at atmospheric pressure and 25° C. The temperature was gradually increased to 85° C. over 15 min, at which point the suspension was about 15° C. above the lipid main-phase transition temperature (65° C. for DAPC) and was removed. The suspension was sonicated with an ultrasonic probe (Model 450, Branson, Danbury, Conn.) for 10 min at 30% power to disperse the lipids into unilamellar vesicles.

Microbubble Production. Size-isolated microbubbles were prepared as previously described. Briefly, 100 mL of lipid suspension was sonicated at low-intensity (30% power) for 10 s. The probe was repositioned at the gas-liquid interface while PFB gas was introduced to the headspace. Microbubbles were produced by high-intensity (100% power) sonication for 10 s. The microbubbles were washed and size-isolated to 4-5 μm by differential centrifugation (Model 5810, Eppendorf, Hauppauge, N.Y.) using PFB-saturated PBS as the processing fluid. Polydisperse microbubbles were prepared by shaking (Amalgamator D-650, TPC, City of Industry, Calif.) a 3-mL serum vial for 45 s with PFB headspace and 2 mL of the same lipid solution as above.

Ligand Conjugation. Azido-functionalized ligands were conjugated to the surface of microbubbles by SPAAC click chemistry. Ligands were dissolved in 1× PBS and mixed with 1 mL of concentrated (3×109 #/mL) microbubbles in a 10:1 ratio of ligands to DSPE-PEG2000-DBCO and allowed to react for 1 h at 25° C. with gentle mixing (end over end). Reaction conditions were adapted from previous work by our group. After conjugation, the microbubbles were washed and concentrated by centrifugation (90 RCF for 1 min).

Microbubble Sizing. Microbubble populations were sized in triplicate before and after surface conjugation by electrozone sensing (Multisizer 3, Beckman Coulter, Indianapolis, Ind.). 0.5 μL of concentrated microbubbles were injected into 10 mL of ISOTON® II diluent and sampled with background subtraction. Number-weighted and volume-weighted particle diameter data were collected in the recommended working range of 2-60% for the 30-μm aperture (0.60-18 μm particle diameter range). Subsequent data analysis was performed with OriginPro (OriginLab, Northampton, Mass.).

Flow Cytometry. Microbubbles were measured before and after Atto 488 conjugation by flow cytometry (Accuri C6, BD Biosciences, San Jose, Calif.). Microbubbles were diluted 100:1 with 1× PBS, and 200 μL was transferred to sampling vial. Samples were run in triplicate with medium fluidics (35 μL/min) and a run limit of 50 μL. Side-scatter (SSC-A), forward-scatter (FSC-A), 533/30 nm filtered light intensity (FL1-A) and 585/40 nm filtered light intensity (FL2-A) were size-gated along the microbubble serpentine pattern, as previously described by Chen et al., and recorded.

Fluorescence Microscopy. Microbubbles reacted with azido-functionalized, photobleach-resistant fluorescein dye (Atto 488) were diluted 100:1 with 1× PBS and 10 μL was pipetted onto a glass slide. Slides were placed on microscope (Model BX52, Olympus, Waltham, Mass.) under low-intensity bright-field light. Microbubble images were focused under 100×, oil-immersion objective in bright-field then imaged by epifluorescence with a 483/31 nm excitation filter and a 535/43 nm emission filter (FITC Filter Cube Set, Edmund Optics, Barrington, N.J.). Images were captured with a digital camera (QIClick Monochrome, Qlmaging, Surrey, BC, Canada) and accompanying software, Q-Capture. Image brightness and contrast post-processing was performed with open-source software ImageJ (NIH, Bethesda, Md.).

Fourier-transform Infrared Spectroscopy. Microbubble shell lipid components, peptide ligands, ligand-conjugated microbubbles and unconjugated microbubbles were analyzed by ATR- FTIR (Cary 630, Agilent, Santa Clara, Calif.). Powdered microbubble shell components (DAPC, DSPE-PEG2000-DBCO and DSPE-PEG5000) and azido-functionalized peptide ligand (A7R and cRGD) samples were analyzed as received from the supplier. Microbubble samples were prepared as described above; however, without size-isolation centrifugation spins 1-mL samples of microbubble cake were collected in 12-mL syringes and separated into three treatment cohorts. SPAAC reactions were performed on two cohorts of microbubble samples, one with azido-functionalized A7R (n=18) and one with cRGD (n=18), as detailed above. The third cohort was not mixed with a reactive ligand species and therefore served as a negative control group (n=15). Conjugated microbubbles were washed three times (90 RCF for 1 min) with deionized water to remove residual salts from the PBS. Resultant microbubble cake from was then transferred to 3-mL serum vials, frozen at −20° C. and lyophilized (FreeZone 1, Labconco Corp., Kansas City, Mo.). Powdered sample absorbance was measured with 32 scans per spectra from 650-4000 cm−1 with resolution of 4 cm−1 at ambient temperature. Spectra were pre-processed using the SNV transform to normalize by sample mass, and processed by Principal Component Analysis (PCA) using the fingerprint region for increased specificity to ligand amino acid groups. SNV transform and PCA analysis was performed with Matlab R2015b software (MathWorks, Inc., Natick, Mass.); SNV transform was performed with a custom script, while PCA analysis was performed with the built-in pca function.

Sterility Assays. To validate microbubble sterility, 1-mL samples of cloaked microbubble samples were tested externally by bacterial endotoxin (BET) analysis (n=3) (Infinity Laboratories, Castle Rock, Colo.) and aerobic bacterial culture (n=4) (Colorado State University Veterinary Diagnostic Laboratories, Fort Collins, Colo.). BET analysis was conducted via kinetic turbidimetric limulus amebocyte lysate (LAL) assay with positive product control in accordance with the United States Pharmacopeia. Aerobic bacterial culture was performed by Colorado State University Veterinary Diagnostic Laboratories (Fort Collins, Colo.). Samples were incubated in applicable growth media for three days where after growth was determined qualitatively.

Canine Tolerability Study. All animal experiments were done with approval of the Institutional Animal Care and Use Committee at Colorado State University. An in vivo dosage escalation tolerability study was conducted with three laboratory beagles (weight=12.5±1.3 kg, no significant change during study). In the three-week study, microbubble dosage was escalated weekly from 1 μL/kg to 100 μL/kg in logarithmic increments, around a target dose of 10 μL/kg, microbubbles at a concentration of 1.0×109 microbubbles/mL. The target dose is the same as prescribed for a commercially available lipid-microbubble formulation, Definity® (Lantheus Medical Imaging, N. Billerica, Mass.). The beagles were placed supine on an examination table, shaved over their left kidney and manually restrained while 7 MHz sagittal-plane CPS ultrasound images were captured using a 15L8-w phased-array transducer and clinical ultrasound scanner (Sequoia C512, Siemens Corp., Washington, D.C.). CPS videos were captured for 20 s prior to microbubble injection, during 2 min low-intensity (PNP=2.12 MPa) observation period, and during 2.5 min high-intensity microbubble elimination period (PNP=5.03 MPa). Microbubbles were injected into the right lateral saphenous vein immediately prior to low-intensity ultrasound imaging observation and followed by a 12-mL saline flush. Videos were captured using Q-Capture (QImaging, Surrey, BC, Canada) software and post-processed using ImageJ to capture specific video frames (NIH, Bethesda, Md.). Serial vital signs (temperature, pulse, respiration) were obtained throughout the day of injection and daily thereafter for 1 week. Clinical pathologic examinations (complete blood count, serum biochemistry profile, coagulation panel) were performed prior to injection and 1, 3 and 7 days following injection. All tests were performed by the Colorado State University Veterinary Diagnostic Laboratories (Fort Collins, Colo.).

### REFERENCES

The following references are hereby incorporated by reference into the specification:

(1) Claudon, M.; Cosgrove, D.; Albrecht, T.; Bolondi, L.; Bosio, M.; Calliada, F.; Correas, J.-M.; Darge, K.; Dietrich, C.; D'Onofrio, M.; et al. Guidelines and Good Clinical Practice Recommendations for Contrast Enhanced Ultrasound (CEUS)—Update 2008. Ultraschall Med.-Eur. J. Ultrasound 2008, 29 (01), 28-44.

(2) Bulte, C. S. E.; Slikkerveer, J.; Meijer, R. I.; Gort, D.; Kamp, O.; Loer, S. A.; Marchi, S. F. de; Vogel, R.; Boer, C.; Bouwman, R. A. Contrast-Enhanced Ultrasound for Myocardial Perfusion Imaging. Anesth. Analg. 2012, 114 (5), 938-945.

(3) Piscaglia, F.; Nols∅m, C.; Dietrich, C. F.; Cosgrove, D. 0.; Gilja, O. H.; Nielsen, M. B.; Albrecht, T.; Barozzi, L.; Bertolotto, M.; Catalano, O.; et al. The EFSUMB Guidelines and Recommendations on the Clinical Practice of Contrast Enhanced Ultrasound (CEUS): Update 2011 on Non-Hepatic Applications. Ultraschall Med.—Eur. J. Ultrasound 2012, 33 (01), 33-59.

(4) Dayton, P. A.; Ferrara, K. W. Targeted Imaging Using Ultrasound. J. Magn. Reson. Imaging 2002, 16 (4), 362-377.

(5) Lindner, J. R. Microbubbles in Medical Imaging: Current Applications and Future Directions. Nat. Rev. Drug Discov. 2004, 3 (6), 527.

(6) Yeh, J. S.-M.; Sennoga, C. A.; McConnell, E.; Eckersley, R.; Tang, M.-X.; Nourshargh, S.; Seddon, J. M.; Haskard, D. O.; Nihoyannopoulos, P. A Targeting Microbubble for Ultrasound Molecular Imaging. PLoS ONE 2015, 10 (7).

(7) Smeenge, M.; Tranquart, F.; Mannaerts, C. K.; de Reijke, T. M.; van de Vijver, M. J.; Laguna, M. P.; Pochon, S.; de la Rosette, J. J. M. C. H.; Wijkstra, H. First-in-Human Ultrasound Molecular Imaging With a VEGFR2-Specific Ultrasound Molecular Contrast Agent (BR55) in Prostate Cancer: A Safety and Feasibility Pilot Study. Invest. Radiol. 2017, 52 (7), 419-427.

(8) Willmann, J. K.; Bonomo, L.; Testa, A. C.; Rinaldi, P.; Rindi, G.; Valluru, K. S.; Petrone, G.; Martini, M.; Lutz, A. M.; Gambhir, S. S. Ultrasound Molecular Imaging With BR55 in Patients With Breast and Ovarian Lesions: First-in-Human Results. J. Clin. Oncol. 2017, 35 (19), 2133-2140.

(9) Klibanov, A. L. Ligand-Carrying Gas-Filled Microbubbles: Ultrasound Contrast Agents for Targeted Molecular Imaging. Bioconjug. Chem. 2005, 16 (1), 9-17.

(10) Agard, N. J.; Prescher, J. A.; Bertozzi, C. R. A Strain-Promoted [3+2] Azide-Alkyne Cycloaddition for Covalent Modification of Biomolecules in Living Systems. J. Am. Chem. Soc. 2004, 126 (46), 15046-15047.

(11) Jewett, J. C.; Bertozzi, C. R. Cu-Free Click Cycloaddition Reactions in Chemical Biology. Chem. Soc. Rev. 2010, 39 (4), 1272-1279.

(12) Debets, M. F.; Berkel, S. S. van; Schoffelen, S.; Rutjes, F. P. J. T.; Hest, J. C. M. van; Delft, F. L. van. Aza-Dibenzocyclooctynes for Fast and Efficient Enzyme PEGylation via Copper-Free (3+2) Cycloaddition. Chem. Commun. 2010, 46 (1), 97-99.

(13) Debets, M. F.; Prins, J. S.; Merkx, D.; Berkel, S. S. van; Delft, F. L. van; Hest, J. C. M. van; Rutjes, F. P. J. T. Synthesis of DIBAC Analogues with Excellent SPAAC Rate Constants. Org. Biomol. Chem. 2014, 12 (27), 5031-5037.

(14) Lindner, J. R.; Dayton, P. A.; Coggins, M. P.; Ley, K.; Song, J.; Ferrara, K.; Kaul, S. Noninvasive Imaging of Inflammation by Ultrasound Detection of Phagocytosed Microbubbles. Circulation 2000, 102 (5), 531-538.

(15) Lindner, J. R.; Coggins, M. P.; Kaul, S.; Klibanov, A. L.; Brandenburger, G. H.; Ley, K. Microbubble Persistence in the Microcirculation During Ischemia/Reperfusion and Inflammation Is Caused by Integrin-and Complement-Mediated Adherence to Activated Leukocytes. Circulation 2000, 101 (6), 668-675.

(16) Janssen, B. J. C.; Huizinga, E. G.; Raaijmakers, H. C. A.; Roos, A.; Daha, M. R.; Nilsson-Ekdahl, K.; Nilsson, B.; Gros, P. Structures of Complement Component C3 Provide Insights into the Function and Evolution of Immunity. Nature 2005, 437 (7058), 505-511.

(17) Noris, M.; Remuzzi, G. Overview of Complement Activation and Regulation. Semin. Nephrol. 2013, 33 (6), 479-492.

(18) Borden, M. A.; Sarantos, M. R.; Stieger, S. M.; Simon, S. I.; Ferrara, K. W.; Dayton, P. A. Ultrasound Radiation Force Modulates Ligand Availability on Targeted Contrast Agents. Mol. Imaging 2006, 5 (3), 139-147.

(19) Borden, M. A.; Zhang, H.; Gillies, R. J.; Dayton, P. A.; Ferrara, K. W. A Stimulus-Responsive Contrast Agent for Ultrasound Molecular Imaging. Biomaterials 2008, 29 (5), 597-606.

(20) Chen, C. C.; Borden, M. A. Ligand Conjugation to Bimodal Poly(Ethylene Glycol) Brush Layers on Microbubbles. Langmuir 2010, 26 (16), 13183-13194.

(21) Chen, C. C.; Borden, M. A. The Role of Poly(Ethylene Glycol) Brush Architecture in Complement Activation on Targeted Microbubble Surfaces. Biomaterials 2011, 32 (27), 6579-6587.

(22) Chen, C. C.; Sirsi, S. R.; Homma, S.; Borden, M. A. Effect of Surface Architecture on In Vivo Ultrasound Contrast Persistence of Targeted Size-Selected Microbubbles. Ultrasound Med. Biol. 2012, 38 (3), 492-503.

(23) Borden, M. A.; Streeter, J. E.; Sirsi, S. R.; Dayton, P. A. In Vivo Demonstration of Cancer Molecular Imaging with Ultrasound Radiation Force and Buried-Ligand Microbubbles. Mol. Imaging 2013, 12 (6), 357-363.

(24) Sirsi, S.; Feshitan, J.; Kwan, J.; Homma, S.; Borden, M. Effect of Microbubble Size on Fundamental Mode High Frequency Ultrasound Imaging in Mice. Ultrasound Med. Biol. 2010, 36 (6), 935-948.

(25) Hoff, L. Acoustic Characterization of Contrast Agents for Medical Ultrasound Imaging; Springer Netherlands: Dordrecht, 2001.

(26) Vos, H. J.; Guidi, F.; Boni, E.; Tortoli, P. Method for Microbubble Characterization Using Primary Radiation Force. IEEE Trans. Ultrason. Ferroelectr. Freq. Control 2007, 54 (7), 1333-1345.

(27) Goertz, D. E.; de Jong, N.; van der Steen, A. F. W. Attenuation and Size Distribution Measurements of Definity™ and Manipulated Definity™ Populations. Ultrasound Med. Biol. 2007, 33 (9), 1376-1388.

(28) Talu, E.; Hettiarachchi, K.; Zhao, S.; Powell, R. L.; Lee, A. P.; Longo, M. L.; Dayton, P. A. Tailoring the Size Distribution of Ultrasound Contrast Agents: Possible Method for Improving Sensitivity in Molecular Imaging. Mol. Imaging 2007, 6 (6), 7290.2007.00034.

(29) Feshitan, J. A.; Chen, C. C.; Kwan, J. J.; Borden, M. A. Microbubble Size Isolation by Differential Centrifugation. J. Colloid Interface Sci. 2009, 329 (2), 316-324.

(30) Miller, J. C.; Pien, H. H.; Sahani, D.; Sorensen, A. G.; Thrall, J. H. Imaging Angiogenesis: Applications and Potential for Drug Development. J. Natl. Cancer Inst. 2005, 97 (3), 172-187.

(31) Perret, G. Y.; Starzec, A.; Hauet, N.; Vergote, J.; Le Pecheur, M.; Vassy, R.; Léger, G.; Verbeke, K. A.; Bormans, G.; Nicolas, P.; et al. In Vitro Evaluation and Biodistribution of a 99mTc-Labeled Anti-VEGF Peptide Targeting Neuropilin-1. Nucl. Med. Biol. 2004, 31 (5), 575-581.

(32) D'Andrea, L. D.; Del Gatto, A.; Pedone, C.; Benedetti, E. Peptide-Based Molecules in Angiogenesis. Chem. Biol. Drug Des. 2006, 67 (2), 115-126.

(33) Starzec, A.; Vassy, R.; Martin, A.; Lecouvey, M.; Di Benedetto, M.; Crépin, M.; Perret, G. Y. Antiangiogenic and Antitumor Activities of Peptide Inhibiting the Vascular Endothelial Growth Factor Binding to Neuropilin-1. Life Sci. 2006, 79 (25), 2370-2381.

(34) Starzec, A.; Ladam, P.; Vassy, R.; Badache, S.; Bouchemal, N.; Navaza, A.; du Penhoat, C. H.; Perret, G. Y. Structure-function Analysis of the Antiangiogenic ATWLPPR Peptide Inhibiting VEGF165 Binding to Neuropilin-1 and Molecular Dynamics Simulations of the ATWLPPR/Neuropilin-1 Complex. Peptides 2007, 28 (12), 2397-2402.

(35) Lum, J. S.; Dove, J. D.; Murray, T. W.; Borden, M. A. Single Microbubble Measurements of Lipid Monolayer Viscoelastic Properties for Small-Amplitude Oscillations. Langmuir 2016, 32 (37), 9410-9417.

(36) Lai, P. Y.; Zhulina, E. B. Structure of a Bidisperse Polymer Brush: Monte Carlo Simulation and Self-Consistent Field Results. Macromolecules 1992, 25 (20), 5201-5207.

(37) Siemion, I. Z.; Kluczyk, A.; Cebrat, M. RGD Peptides. In Handbook of biologically active peptides; Kastin, A. J., Ed.; Elsevier/AP: Amsterdam, 2013; pp 705-714.

(38) Perret, G. Y.; Starzec, A.; Hauet, N.; Vergote, J.; Le Pecheur, M.; Vassy, R.; Léger, G.; Verbeke, K. A.; Bormans, G.; Nicolas, P.; et al. In Vitro Evaluation and Biodistribution of a 99mTc-Labeled Anti-VEGF Peptide Targeting Neuropilin-1. Nucl. Med. Biol. 2004, 31 (5), 575-581.

(39) Starzec, A.; Vassy, R.; Martin, A.; Lecouvey, M.; Di Benedetto, M.; Crépin, M.; Perret, G. Y. Antiangiogenic and Antitumor Activities of Peptide Inhibiting the Vascular Endothelial Growth Factor Binding to Neuropilin-1. Life Sci. 2006, 79 (25), 2370-2381.

(40) Song, K.-H.; Fan, A. C.; Hinkle, J. J.; Newman, J.; Borden, M. A.; Harvey, B. K. Microbubble Gas Volume: A Unifying Dose Parameter in Blood-Brain Barrier Opening by Focused Ultrasound. Theranostics 2017, 7 (1), 144-152.

(41) Satinover, S. J.; Dove, J. D.; Borden, M. A. Single-Particle Optical Sizing of Microbubbles. Ultrasound Med. Biol. 2014, 40 (1), 138-147.

(42) Rychak, J. J.; Lindner, J. R.; Ley, K.; Klibanov, A. L. Deformable Gas-Filled Microbubbles Targeted to P-Selectin. J. Control. Release Off. J. Control. Release Soc. 2006, 114 (3), 288-299.

(43) Borden, M. A.; Martinez, G. V.; Ricker, J.; Tsvetkova, N.; Longo, M.; Gillies, R. J.; Dayton, P. A.; Ferrara, K. W. Lateral Phase Separation in Lipid-Coated Microbubbles. Langmuir ACS J. Surf. Colloids 2006, 22 (9), 4291-4297.

(44) Fearn, T.; Riccioli, C.; Garrido-Varo, A.; Guerrero-Ginel, J. E. On the Geometry of SNV and MSC. Chemom. Intell. Lab. Syst. 2009, 96 (1), 22-26.

(45) Dehghani-Bidgoli, Z.; Baygi, M. H. M.; Kabir, E.; Malekfar, R. Developing an Instrument-Independent Algorithm for Raman Spectroscopy: A Case of Cancer Detection. Technol. Cancer Res. Treat. 2014, 13 (2), 119-127.

(46) Fringeli, U. P.; Müldner, H. G.; Günthard, H. H.; Gasche, W.; Leuzinger, W. The Structure of Lipids and Proteins Studied by Attenuated Total-Reflection (ATR) Infrared Spectroscopy. Z. Für Naturforschung B 1972, 27 (7).

(47) Deygen, I. M.; Kudryashova, E. V. New Versatile Approach for Analysis of PEG Content in Conjugates and Complexes with Biomacromolecules Based on FTIR Spectroscopy. Colloids Surf. B Biointerfaces 2016, 141, 36-43.

(48) Barth, A. The Infrared Absorption of Amino Acid Side Chains. Prog. Biophys. Mol. Biol. 2000, 74 (3), 141-173.

(49) Thamm, D. H.; Vail, D. M. Veterinary Oncology Clinical Trials: Design and Implementation. Vet. J. 2015, 205 (2), 226-232.

(50) United States Pharmacopeia. Chapter <85>, Bacterial Endotoxin Tests. In USP 39; U.S. Pharmacopeial Convention: Rockville, Md., 2015; pp 161-167.

(51) Garg, S.; Thomas, A. A.; Borden, M. A. The Effect of Lipid Monolayer In-Plane Rigidity on in Vivo Microbubble Circulation Persistence. Biomaterials 2013, 34 (28), 6862-6870.

(52) Chen, C. C. Engineering Microbubbles with the Buried-Ligand Architecture for Targeted Ultrasound Molecular Imaging, Columbia University, 2011.

(53) Agard, N. J.; Prescher, J. A.; Bertozzi, C. R. A Strain-Promoted [3+2] Azide-Alkyne Cycloaddition for Covalent Modification of Biomolecules in Living Systems. J. Am. Chem. Soc. 2004, 126 (46), 15046-15047.

(54) Satinover, S. J.; Dove, J. D.; Borden, M. A. Single-Particle Optical Sizing of Microbubbles. Ultrasound Med. Biol. 2014, 40 (1), 138-147.

(55) Barnes, R. J.; Dhanoa, M. S.; Lister, S. J. Standard Normal Variate Transformation and De-Trending of near-Infrared Diffuse Reflectance Spectra. Appl. Spectrosc. 1989, 43 (5), 772-777.

What is claimed is:

1-24. (canceled)

25. A conjugated microbubble comprising:
a polymer tether coupled with the surface of a microbubble;
at least one azido-functionalized ligand conjugated with said polymer tether through a click chemistry reaction to form a bioconjugate polymer.

26. The conjugated microbubble of claim 25 wherein said at least one azido-functionalized ligand conjugated with said polymer tether through a click chemistry reaction to form a bioconjugate polymer comprises at least one azido-functionalized ligand conjugated with said polymer tether through a process of strain-promoted [3+2] azide-alkyne cycloaddition (SPAAC).

27. The conjugated microbubble of claim 25 wherein said azido-functionalized ligand conjugated with said polymer tether through a click chemistry reaction to form a bioconjugate polymer comprises least one azido-functionalized ligand conjugated with said polymer tether through a strain-promoted [3+2] azide-alkyne cycloaddition (SPAAC) conjugation reaction between a polymer tether comprising PEGylated, dibenzocyclooctyne-functionalized phosphatidylethanolamine (DSPE-PEG2000-DBCO), and said azido-functionalized peptide ligand to form 1,2,3-triazole linked bioconjugate, wherein said SPAAC reaction is performed in the absence of a copper (Cu) catalyst.

28. The conjugated microbubble of claim 27 wherein said microbubble comprises a microbubble having buried-ligand architecture (BLA).

29. The conjugated microbubble of claim 28 wherein said microbubble having BLA comprises a microbubble having hydrated polymer brush architecture.

30. The conjugated microbubble of claim 29 wherein said microbubble having hydrated polymer brush architecture comprises a microbubble having bimodal PEGylated surface architecture.

31. The conjugated microbubble of claim 30 wherein said microbubble having bimodal PEGylated surface architecture comprises a microbubble having:
a plurality of shorter polyethylene glycol (PEG) molecule forming said polymer tether that attaches said azido-functionalized ligand to an anchoring lipid on said microbubble; and
a plurality of longer PEG chains that stratify into an overbrush that cloaks said azido-functionalized ligand.

32. The conjugated microbubble of claim 31 wherein said shorter PEG molecule forming said polymer tether has a molecular weight of ˜2000 Dalton (Da), and said longer PEG chains forming said overbrush has a molecular weight of ˜5000 Da.

33. The conjugated microbubble of claim 26 wherein said azido-functionalized ligand is cloaked by BLA on said microbubble.

34. The conjugated microbubble of claim 33 wherein said azido-functionalized ligand comprises at least one azido-functionalized biomarker ligand.

35. The conjugated microbubble of claim 34 wherein said at least one azido-functionalized biomarker ligand comprises at least one azido-functionalized angiogenesis biomarker ligand.

36. The conjugated microbubble of claim 35 wherein said at least one azido-functionalized angiogenesis biomarker ligand comprises an azido-functionalized integrin αvβ3 antagonist (cRGD) ligand.

37. The conjugated microbubble of claim 35 wherein said at least one azido-functionalized angiogenesis biomarker ligand comprises an azido-functionalized VEGFR2 antagonist (A7R) ligand.

38. The conjugated microbubble of claim 34 wherein said at least one azido-functionalized biomarker ligand comprises at least one of the following
at least one azido-functionalized cancer biomarker ligand;
at least one azido-functionalized therapeutic ligand; and
at least one azido-functionalized diagnostic ligand.

39-40. (canceled)

41. The conjugated microbubble of claim 26 wherein said conjugated microbubble is generated aseptically, and/or said conjugated microbubble is aseptic.

42. (canceled)

43. The conjugated microbubble of claim 33 and further comprising administering a therapeutically effective amount of the cloaked microbubble to a patient in need thereof.

44. The conjugated microbubble of claim 43 wherein said azido-functionalized ligand is transiently revealed through applying ultrasound radiation to said cloaked microbubble.

45. (canceled)

46. The conjugated microbubble of claim 45 wherein said cloaked microbubble is between 3-7 μm in diameter.

47. A conjugated microbubble comprising:
a polymer tether coupled with the surface of a microbubble; and
at least one azido-functionalized ligand conjugated with said polymer tether through a process of strain-promoted [3+2] azide-alkyne cycloaddition (SPAAC) to form a bioconjugate polymer.

48. A conjugated microbubble comprising:
a polymer tether coupled with the surface of a microbubble, wherein said polymer tether is PEGylated, dibenzocyclooctyne-functionalized phosphatidylethanolamine (DSPE-PEG2000-DBCO); and
at least one azido-functionalized peptide ligand conjugated with said polymer tether through a process of strain-promoted [3+2] azide-alkyne cycloaddition (SPAAC), wherein said SPAAC reaction is performed in the absence of a copper (Cu) catalyst.
